(19) United States
(12) Patent Application Publication
(10) Pub. No.: US 2026/0315809 A1
(43) Pub. Date: Oct. 15, 2026

(54) SURROGATE LEFT VENTRICULAR ACTIVATION TIME

(71) Applicant: Medtronic, Inc., Minneapolis, MN (US)

(72) Inventors: Hongyang Lu, Shanghai (CN); Tianyi Shi, Shanghai (CN); Jian Cao, Minneapolis, MN (US); Xiaohong Zhou, Minneapolis, MN (US)

(21) Appl. No.: 19/104,493

(22) PCT Filed: Aug. 24, 2022
(86) PCT No.: PCT/CN2022/114465

§ 371 Date: Feb. 18, 2025

(51) Int. Cl. A61N 1/37; A61N 1/05; A61N 1/365; A61N 1/368

(52) U.S. Cl. CPC A61N 1/3712; A61N 1/056; A61N 1/36507; A61N 1/3686

(57) ABSTRACT

The present disclosure relates to surrogate left ventricular activation times (110, 107, 107A) that is representative of, or correlates to, left ventricular activation times for use in determining left bundle branch (LBB) (8a) capture and LBB pacing configuration. The surrogate left ventricular activation times (110, 107, 107A) may be determined, or measured, from electrical activity monitored from one or more implanted electrodes (40, 42, 44, 46, 48, 50, 58, 61, 62, 64, 66) such as, for example, a LBB pacing electrode or an electrode located in the right ventricle (28).

The present disclosure relates generally to determining left bundle branch capture using electrogram signal analysis to, for example, ensure capture of the left bundle branch as opposed to capture of myocardial tissues of the heart. More particularly, the present disclosure relates to electrogram signal analysis as opposed to electrocardiogram signal analysis, which, for example, may advantageously provide a more efficient means for determining left bundle branch capture, or may advantageously negate the need to travel to a clinic.

Implantable medical devices (IMDs), such as cardiac pacemakers or implantable cardioverter defibrillators, deliver therapeutic stimulation to patients' hearts thereby improving the lives of millions of patients living with heart conditions. Conventional pacing techniques involve pacing one or more of the four chambers of a patient's heart 12 as illustrated in FIG. 1, including left atrium (LA) 33, right atrium (RA) 26, left ventricle (LV) 32 and right ventricle (RV) 28. One common conventional therapeutic pacing technique that treats a slow heart rate, referred to as Bradycardia, involves delivering an electrical pulse to a patient's right ventricular tissue. In response to the electrical pulse, both the right and left ventricles contract. However, the heart beat process may be significantly delayed because the pulse travels from the right ventricle through the left ventricle. The electrical pulse passes through the muscle cells that are referred to as myocytes. Myocyte-to-myocyte conduction may be very slow. Delayed electrical pulses can cause the left ventricle to be unable to maintain synchrony with the right ventricle.

Over time, the left ventricle can become significantly inefficient at pumping blood to the body. In some patients, heart failure can develop such that the heart is too weak to pump blood to the body. Heart failure may be a devastating diagnosis since, for example, fifty percent of the heart failure patients have a life expectancy of five years. Another possible cause of heart failure is due to atrial fibrillation, which is an irregular and often very rapid heart rhythm or arrhythmia. During atrial fibrillation, for example, the atria of the heart can beat out of sync with the ventricles of the heart because of the arrythmia of the atria, and this can lead to issues such as blood clots in the heart or increase the risk of stroke or heart failure. To avoid the potential development of heart failure, some physicians have considered alternative pacing methods that involve the cardiac conduction system. The cardiac conduction system may be described as being able to quickly conduct electrical pulses (for example, akin to a car driving on a highway), whereas pacing cardiac muscle (myocardial) tissue may more slowly conduct electrical pulses (for example, akin to a car driving on a dirt road).

The cardiac conduction system includes sinoatrial node (SA node) 1, atrial internodal tracts 2, 4, 5 (i.e., anterior internodal 2, middle internodal 4, and posterior internodal 5), atrioventricular node (AV node) 3, His bundle 13 (also known as atrioventricular bundle or bundle of His), and left and right bundle branches 8a, 8b. FIG. 1 also shows the arch of aorta 6 and Bachman's bundle 7. The SA node, located at the junction of the superior vena cava and right atrium, is considered to be the natural pacemaker of the heart since it continuously and repeatedly emits electrical impulses. The electrical impulse spreads through the muscles of right atrium 26 to left atrium 33 to cause synchronous contraction of the atria. Electrical impulses are also carried through atrial internodal tracts to atrioventricular (AV) node 3—the sole connection between the atria and the ventricles. Conduction through the AV nodal tissue takes longer than through the atrial tissue, resulting in a delay between atrial contraction and the start of ventricular contraction. The AV delay, which is the delay between atrial contraction and ventricular contraction, allows the atria to empty blood into the ventricles. Then, the valves between the atria and ventricles close before causing ventricular contraction via branches of the bundle of His. His bundle 13 is located in the membranous atrioventricular septum near the annulus of the tricuspid valve. His bundle 13 splits into left and right bundle branches 8a, 8b and are formed of specialized fibers called “Purkinje fibers” 9. Purkinje fibers 9 may be described as rapidly conducting an action potential down the ventricular septum (VS), spreading the depolarization wavefront quickly through the remaining ventricular myocardium, and producing a coordinated contraction of the ventricular muscle mass.

While cardiac conduction system pacing therapy is increasingly used as an alternative to traditional pacing techniques, cardiac conduction system pacing therapy has not been widely adopted for a variety of reasons. For example, cardiac conduction system pacing electrodes should be positioned within precise target locations (e.g., within about 1 millimeter) of portions or regions of the cardiac conduction system, such as the His bundle, which may be difficult. Additionally, adjustment of cardiac conduction system pacing therapy during delivery of therapy may be challenging. Further, determination of whether the cardiac conduction system pacing therapy is selective (i.e., only pacing the cardiac conduction system) or non-selective (i.e., pacing both the cardiac conduction system and the myocardial tissue) may also be challenging. It is desirable to develop new cardiac conduction system pacing therapy systems, devices, and methods and systems that overcome some of the disadvantages associated with previously-performed cardiac conduction system pacing therapies.

### SUMMARY

This disclosure generally relates to determining left bundle branch (LBB) capture using electrogram (EGM) signal analysis, for example, to ensure capture of the LBB as opposed to capture of myocardial tissues of the heart. LBB pacing is a physiological pacing approach, and LBB capture is required for such an approach. Conversely, left ventricular (LV) septal pacing captures and paces at the septum, which is not part of the cardiac conduction system. It can be difficult to implant a lead close enough to the LBB to effectively pace the LBB, or implanted LBB lead(s) may dislodge over time due to natural movement or due to injury, for example, and LV septal pacing may occur as a result. To distinguish LBB pacing from LV septal pacing and ensure capture of the LBB, a standard 12-lead electrocardiogram (ECG) is typically used, and the resulting ECG signal(s) are analyzed. Typically, the resulting ECG is used to determine left ventricular (LV) activation time in order to distinguish LBB pacing from LV septal pacing. If the LV activation time remains short and constant at different pacing outputs, it is generally concluded that the LBB is captured as opposed to capture of myocardial tissues of the heart.

In particular, illustrative devices and methods are described herein to determine LBB capture using EGM signal analysis (as opposed to ECG signal analysis) and a determined surrogate LV activation time based on the EGM signal analysis. Use of EGM signals as opposed to ECG signals, for example, may advantageously provide more efficient or more effective analysis, and may negate the need for a patient to visit a clinic to have ECG signals measured. Surrogate LV activation time is detected from cardiac conduction system pacing pulse (e.g., LBB pacing pulse) to a sensed ventricular event (e.g., RV depolarization as measured using electrical signal QRS morphology, etc.).

The surrogate LV activation time may be used to determine effective LBB capture and effective LBB pacing using a variety of methods and applications as described herein. In one or more embodiments, illustrative devices and methods are described herein to periodically determine that effective LBB pacing therapy is being delivered, as opposed to LV septal pacing, for example, so as to provide effective cardiac therapy to a patient over time. Effective LBB capture and LBB pacing may be desirable for a patient undergoing cardiac resynchronization therapy (CRT), for example, or may be desirable to ensure capture of the patient's right ventricle (RV), for another example.

The illustrative devices and methods may be described as utilizing at least a single-chamber device solution for LBB pacing therapy-indicated patients that may include one or more of a standard right atrial lead, a 3830 or 3830 D lead for cardiac conduction system pacing, and a left ventricular lead.

One illustrative implantable medical device may comprise one or more implantable electrodes. The one or more implantable electrodes may comprise an LBB electrode. The LBB electrode may be positionable proximate a portion of the patient's left bundle branch (LBB). The implantable medical device may further comprise a computing apparatus. The computing apparatus may comprise processing circuitry. The computing apparatus may be operably coupled to the one or more implantable electrodes. The computing apparatus may be configured to deliver LBB pacing proximate to the LBB using the LBB electrode at one or more pacing configurations. The computing apparatus may be further configured to monitor electrical activity using the one or more implantable electrodes during delivery of the LBB pacing at the one or more pacing configurations. The computing apparatus may be further configured to determine a surrogate LV activation time based on the monitored electrical activity for each of the one or more pacing configurations. The surrogate LV activation time may be representative of an LV activation time. The computing apparatus may be further configured to determine whether the LBB pacing has captured the LBB for each of the one or more pacing configurations based on the determined surrogate LV activation time for each of the one or more pacing configurations.

One illustrative method may include delivering LBB pacing proximate to a patient's left bundle branch (LBB). Such delivery of LBB pacing may be done using an LBB electrode positioned proximate a portion of the LBB. Such delivery of LBB pacing may be done at one or more pacing configurations. The method may further include monitoring electrical activity using one or more electrodes during delivery of the LBB pacing at the one or more pacing configurations. The method may further include determining a surrogate LV activation time based on the monitored electrical activity for each of the one or more pacing configurations. The surrogate LV activation time may be representative of an LV activation time. The method may further include determining whether the LBB pacing has captured the LBB for each of the one or more pacing configurations based on the determined surrogate LV activation time for each of the one or more pacing configurations.

The above summary is not intended to describe each embodiment or every implementation of the present disclosure. A more complete understanding will become apparent and appreciated by referring to the following detailed description and claims taken in conjunction with the accompanying drawings.

### BRIEF DESCRIPTION OF THE DRAWINGS

FIG. 1 is a schematic diagram of a heart of patient.

FIG. 2A is a conceptual diagram illustrating an example of a therapy system (e.g., triple-chamber implantable medical device) that is configured to provide therapy to a heart of patient through a His-bundle or bundle-branch pacing lead and lead placed either in the right ventricle or the right atrium using an implantable medical device (IMD).

FIG. 2B is a schematic diagram illustrating an example of a His-bundle or bundle-branch pacing lead positioned in bundle of the His in a cross-sectional view of the heart.

FIG. 3A is a conceptual diagram illustrating an example therapy system (e.g., dual-chamber implantable medical device) that includes a His-bundle or bundle-branch pacing lead and lead placed in the left ventricle using an IMD.

FIG. 3B is a schematic diagram illustrating an example of a His-bundle or bundle-branch pacing lead positioned in bundle of the His in a cross-sectional view of the heart using an IMD.

FIG. 3C is a cross-sectional view of a patient's heart implanted with an implantable medical electrical lead to deliver bundle branch pacing.

FIG. 3D is a close-up view of the lead in the patient's heart of FIG. 3C.

FIG. 4 is a conceptual diagram illustrating an example of a therapy system (e.g., dual chamber implantable medical device) that is configured to provide therapy to a heart of patient through a His-bundle or bundle-branch pacing lead and lead placed in the left ventricle using an IMD.

FIG. 5 is a functional block diagram illustrating an example of a configuration of an implantable medical device of FIG. 2A and 3A-4.

FIG. 6 is a block diagram of an illustrative method of determining effective LBB capture and LBB pacing based on monitored electrical activity that may be utilized by the devices of FIGS. 2-5.

FIG. 7 is a depiction of exemplary ECG and EGM signals over time illustrating determination of a surrogate LV activation time of the method of FIG. 6.

FIG. 8 is a scatter plot illustrating a correlation between a surrogate LV activation time determined as shown in FIG. 7 and a measured LV activation time.

FIG. 9 is another depiction of exemplary ECG and EGM signals over time, illustrating determination of a surrogate LV activation time of the method of FIG. 6.

FIG. 10 is a scatter plot illustrating a correlation between another, different surrogate LV activation time determined as shown in FIG. 9 and LV activation time.

FIG. 11 is a block diagram of an illustrative method of determining LBB capture using an EGM signal that may be utilized by the devices, methods, and processes of FIGS. 2-7 and 9.

FIG. 12 is a block diagram of another illustrative method of determining LBB capture using an EGM signal that may be utilized by the devices, methods, and processes of FIGS. 2-7 and 9.

FIG. 13 is a scatter plot illustrating correlated surrogate LV activation times and LVATs at various pacing outputs indicating effective LBB capture.

FIG. 14 is a scatter plot illustrating correlated surrogate LV activation times and LVATs at various pacing outputs indicating ineffective LBB capture.

### DETAILED DESCRIPTION

In the following detailed description of illustrative embodiments, reference is made to the accompanying figures of the drawing which form a part hereof, and in which are shown, by way of illustration, specific embodiments which may be practiced. It is to be understood that other embodiments may be utilized, and structural changes may be made without departing from (e.g., still falling within) the scope of the disclosure presented hereby.

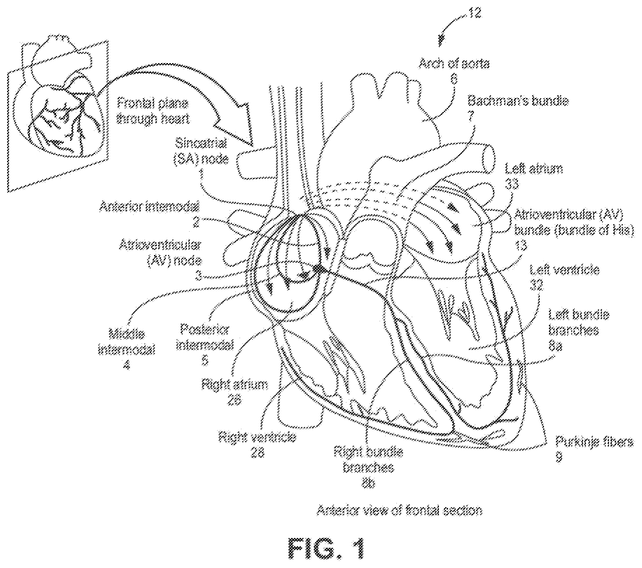

Illustrative systems, devices, and methods shall be described with reference to FIGS. 1-12. It will be apparent to one skilled in the art that elements or processes from one embodiment may be used in combination with elements or processes of the other embodiments, and that the possible embodiments of such systems, devices, and methods using combinations of features set forth herein is not limited to the specific embodiments shown in the Figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure, although certain timings, one or more shapes and/or sizes, or types of elements, may be advantageous over others.

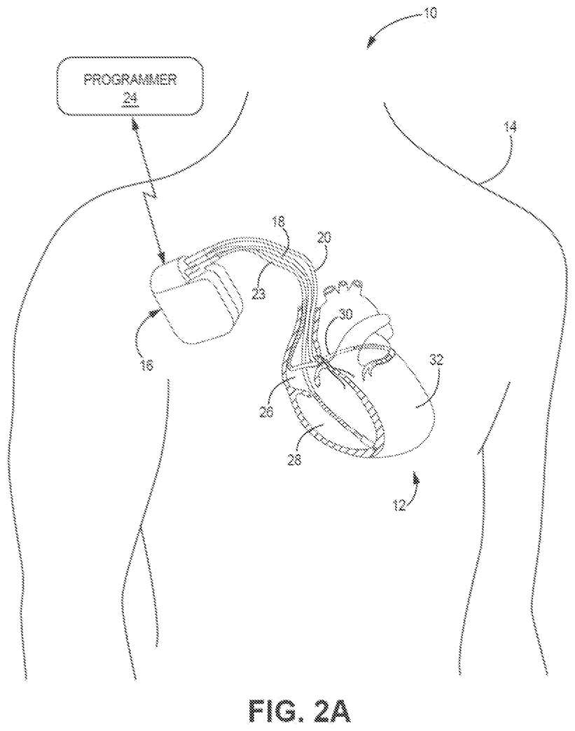

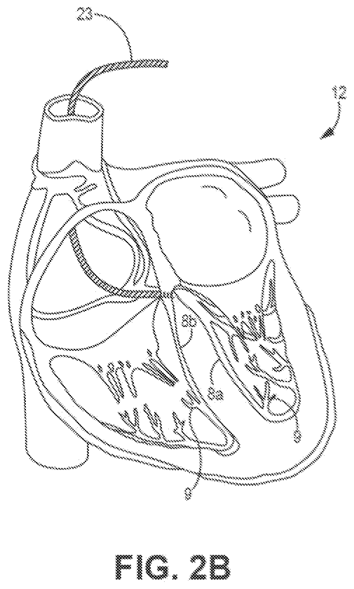

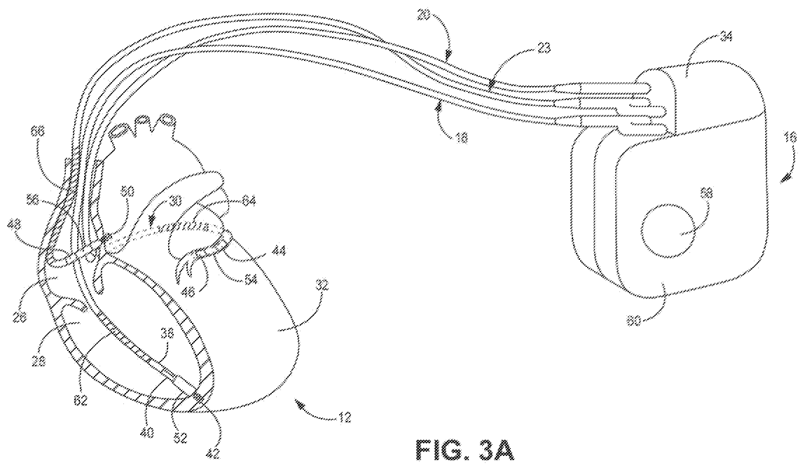

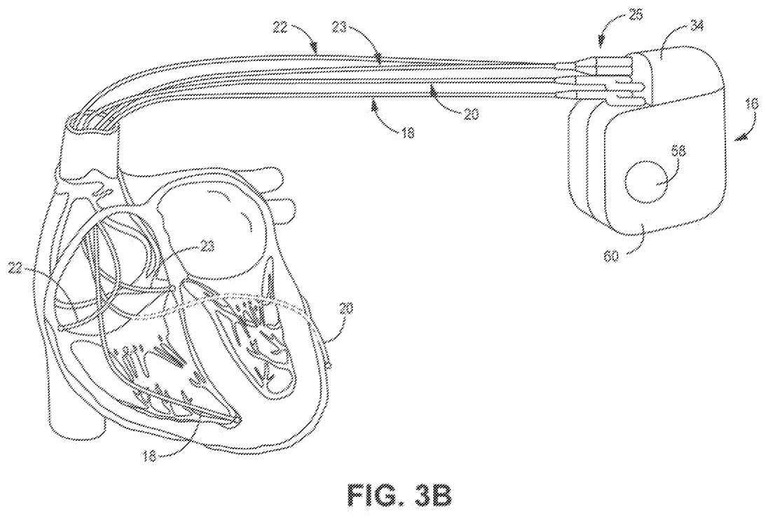

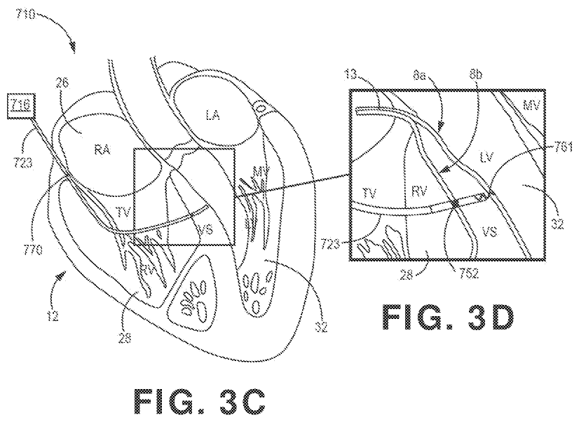

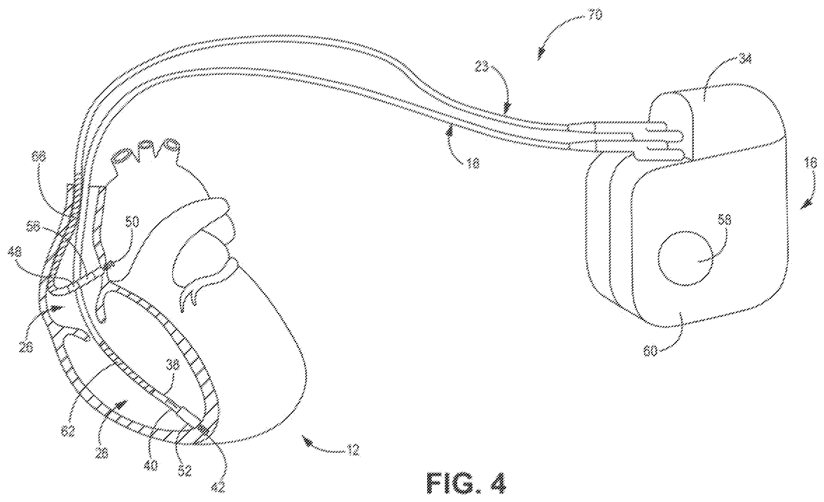

FIG. 1 is a schematic diagram of heart 12. FIGS. 2A and 3A are conceptual diagrams illustrating one example therapy system 10 that may be used to provide therapy to heart 12 of patient 14. Patient 14 ordinarily, but not necessarily, will be a human. Therapy system 10 includes IMD 16, which is coupled to leads 18, 20, 23 and programmer 24. The lead 23 is shown in isolation, or by itself, in FIG. 2B in relation to the bundle branches 8a, 8b and Purkinje fibers 9 of the heart 12. IMD 16 may be, for example, an implantable pacemaker, cardioverter, and/or defibrillator that provides electrical signals to heart 12 via electrodes coupled to one or more of leads 18, 20, 23. Further non-limiting examples of IMD 16 include: a pacemaker with a medical lead, an implantable cardioverter-defibrillator (ICD), an intracardiac device, a leadless pacing device (LPD), a subcutaneous ICD (S-ICD), and a subcutaneous medical device (e.g., nerve stimulator, inserted monitoring device, etc.).

Leads 18, 20, 23 extend into heart 12 of patient 14 to sense electrical activity of heart 12 and/or deliver electrical stimulation to heart 12. In the example shown in FIG. 2A, right ventricular (RV) lead 18 extends through one or more veins (not shown), the superior vena cava (not shown), and right atrium (RA) 26, and into right ventricle 28. Left ventricular (LV) coronary sinus lead 20 extends through one or more veins, the vena cava, right atrium 26, and into the coronary sinus 30 to a region adjacent to the free wall of left ventricle 32 of heart 12. Cardiac conduction system pacing therapy lead 23 (e.g., left bundle branch pacing lead, right bundle branch pacing lead, His-bundle pacing lead, etc.) extends through one or more veins and the vena cava, and into the right atrium 26 of heart 12 to pace the cardiac conduction system (e.g., triangle of Koch, septal wall, left bundle branch, right bundle branch, the His bundle, etc.). In some embodiments, the cardiac conduction system pacing therapy lead 23 may be positioned within about 1 millimeter of the triangle of Koch, septal wall, His bundle, or one or both bundle branches.

As used herein, cardiac conduction system pacing therapy refers to any pacing therapy configured to deliver pacing therapy (e.g., pacing pulses) to the cardiac conduction system including, e.g., the His bundle, left bundle branch, right bundle branch, etc. As used herein, the term “activation” refers to a sensed or paced event. For example, an atrial activation may refer to an atrial sense or event (As) or an atrial pace or artifact of atrial pacing (Ap). Similarly, a ventricular activation may refer to a ventricular sense or event (Vs) or a ventricular pace or artifact of ventricular pacing (Vp), which may be described as ventricular stimulation pulses. In some embodiments, activation interval, or “activation time” can be detected from As or Ap to Vs or Vp, as well as Vp to Vs. In particular, activation time may include a pacing (Ap or Vp) to ventricular interval (LV or RV sense) or an atrial-sensing (As) to ventricular-sensing interval (LV or RV sense). Still further, activation time may include a cardiac conduction system pacing pulse (e.g., LBB pacing pulse) to ventricular interval such as breakout ventricular myocardial depolarization (RV sense). Surrogate LV activation time is detected from cardiac conduction system pacing pulse (e.g., LBB pacing pulse) to a sensed ventricular event (e.g., RV depolarization as measured using electrical signal QRS morphology, etc.).

IMDs may be described as delivering one or both of conventional pacing therapy and cardiac conduction system pacing therapy. Conventional, or traditional, pacing therapy may be described as delivering pacing pulses into myocardial tissue that is not part of the cardiac conduction system of the patient's heart such that, e.g., the pacing pulses trigger electrical activation that propagates primarily from one myocardial cell to another myocardial cell (also referred to as “cell-to-cell”) as opposed to propagating within the cardiac conduction system prior to the myocardial tissue. For instance, conventional pacing therapy may deliver pacing pulses directly into the muscular heart tissue that is to be depolarized to provide the contraction of the heart. For example, conventional left ventricular pacing therapy may utilize a left ventricular (LV) coronary sinus lead that is implanted so as to extend through one or more veins, the vena cava, the right atrium, and into the coronary sinus to a region adjacent to the free wall of the left ventricle of the heart so as to deliver pacing pulses to the myocardial tissue of the free wall of the left ventricle.

One example of a cardiac conduction system pacing therapy lead 23 (e.g., a His lead) can be the SELECTSURE™ 3830. A description of the SELECTSURE™ 3830 is found in the Medtronic model SELECTSURE™ 3830 manual (2013), incorporated herein by reference in its entirety. The SELECTSURE™ 3830 includes two or more conductors with or without lumens.

An exemplary left ventricular lead with a set of spaced apart electrodes is shown in US Pat. Pub. No. WO 2019/104174 A1, filed on May 4, 2012, by Ghosh et al., commonly assigned by the assignee of the present disclosure, the disclosure of which is incorporated by reference in its entirety herein. Exemplary electrodes on leads to form pacing vectors are shown and described in U.S. Pat. Nos. 8,355,784 B2, 8,96S,5G7, and 8,126,546, all of which are incorporated by reference and can implement features of the disclosure.

Illustrative cardiac conduction system pacing therapy may be described in, for example, U.S. Pat. App. Pub. No. 2019/0111270 A1 entitled “His Bundle and Bundle Branch Pacing Adjustment” published on Apr. 18, 2019, which is incorporated herein by reference in its entirety.

An elongated conductor of the lead may extend through a hermetic feedthrough assembly, and within an insulative tubular member of the lead, and may electrically couple an electrical pulse generator (contained within housing) to the helical tip electrode, or cardiac conduction system electrode, of the cardiac conduction system pacing therapy lead 23. The conductor may be formed by one or more electrically conductive wires, for example, MP35N alloy known to those skilled in the art, in a coiled or cabled configuration, and the insulative tubular member may be any suitable medical grade polymer, for example, polyurethane, silicone rubber, or a blend thereof. According to an illustrative embodiment, the flexible lead body extends a pre-specified length (e.g., about 10 centimeters (cm) to about 20 cm, or about 15 to 20 cm) from a proximal end of housing to the other end. The lead body is less than about 7 French (FR) but typically in the range of about 3 to 4 FR in size. In one or more embodiments, about 2 to about 3 FR size lead body is employed.

Cardiac conduction system pacing therapy can be performed by other leads. Another illustrative lead, including two or more pacing electrodes, can be used to deliver multisite pacing pulses to the cardiac conduction system via the bundle of His or one or both bundle branches. Multisite pacing can be delivered simultaneously or sequentially, as described and shown by U.S. Pat. App. Pub. No. 2016/0339248, filed on Apr. 21, 2016, entitled EFFICIENT DELIVERY OF MULTI-SITE PACING, the disclosure of which is incorporated by reference in its entirety.

Since the electrodes in multi-site or multi-point stimulation may be in close proximity to each other, it may be important to detect and verify effective capture of individual electrodes during delivery of such therapy. Delivering multisite pacing pulses may include delivering pacing pulses to a first tissue site and a second tissue site through first and second pacing electrodes, respectively, all of which may occur within the same cardiac cycle.

In particular, a lead configured to perform multi-site pacing, which is different than LV coronary sinus lead 20, can be placed in the ventricular septum with the first (distal) electrode towards the left side of the ventricular septum for left bundle branch pacing and with the second electrode (proximal) towards the right side of the septum for pacing the right bundle branch. An interelectrode distance may be defined as the distance between the first and second electrodes, or the distance that the electrodes are apart. In some embodiments, the interelectrode distance is at least about 3, 4, 5, 6, 7, or 8 millimeters (mm). In some embodiments, the interelectrode distance is at most about 15, 14, 13, 12, 11, or 10 mm. For example, the interelectrode distance may be in a range from about 6 to 12 mm apart. Once the pacing is delivered, both the left bundle branch and the right bundle branch may be stimulated such that both ventricles are naturally or near-naturally synchronized. In contrast, in traditional CRT, the ventricles may be described as not naturally synchronized.

A single lead, including two (or more) pacing electrodes (e.g., cathodes) may deliver cathode pacing outputs at two separate locations (e.g., left and right bundle branches), so both bundle branches can be excited at the same time.

Cardiac conduction system pacing may include at least one of His bundle or left or right bundle branch pacing. Bundle branch pacing may bypass the pathological region and may have a low and stable pacing threshold. In some embodiments, only one bundle branch may be paced by using pacing leads. In further embodiments, both bundle branches may be paced at the same time (e.g., dual bundle branch pacing), which may mimic intrinsic activation propagation via the His bundle-Purkinje conduction system, e.g., paced activation propagates via both bundle branches to both ventricles for synchronized contraction. Traditional His bundle pacing, on the other hand, typically paces the His bundle proximal to the bundle branches. In some embodiments, IMD 16 may include one, two, or more electrodes located in one or more bundle branches configured for bundle branch pacing. In some embodiments, IMD 16 may be an intracardiac pacemaker or leadless pacing device (LPD).

As used herein, “leadless” refers to a device being free of a lead extending out of patient's heart 12. In other words, a leadless device may have a lead that does not extend from outside of the patient's heart to inside of the patient's heart. Some leadless devices may be introduced through a vein, but once implanted, the devices are free of, or may not include, any transvenous lead and may be configured to provide cardiac therapy without using any transvenous lead. In one or more embodiments, an LPD for bundle pacing does not use a lead to operably connect to an electrode disposed proximate to the septum when a housing of the device is positioned in the atrium. A leadless electrode may be leadlessly coupled to the housing of the medical device without using a lead between the electrode and the housing.

IMD 16 may sense electrical signals attendant to the depolarization and repolarization of heart 12 via electrodes (not shown in FIGS. 2A-B) coupled to at least one of leads 18, 20, 23. In some examples, IMD 16 provides pacing pulses to heart 12 based on the electrical signals sensed within heart 12. The configurations of electrodes used by IMD 16 for sensing and pacing may be unipolar or bipolar. IMD 16 may also provide defibrillation therapy and/or cardioversion therapy via electrodes located on at least one of leads 18, 20, 23. IMD 16 may detect atrial arrhythmias of heart 12, such as atrial fibrillation of atria 26 and 33, and may deliver defibrillation therapy to heart 12 in the form of electrical pulses. Also, IMD 16 may detect ventricular arrhythmias of heart 12, such as ventricular fibrillation of ventricles 28 and 32, and may deliver defibrillation therapy to heart 12 in the form of electrical pulses. In some examples, IMD 16 may be programmed to deliver a progression of therapies, e.g., pulses with increasing energy levels, until a fibrillation of heart 12 is stopped. IMD 16 may detect fibrillation employing one or more fibrillation detection techniques known in the art.

In some examples, programmer 24 (FIG. 1) may be a handheld computing device or a computer workstation or a mobile phone. Programmer 24 may include a user interface that receives input from a user. The user interface may include, for example, a keypad and a display, which may for example, be a cathode ray tube (CRT) display, a liquid crystal display (LCD) or light emitting diode (LED) display. The keypad may take the form of an alphanumeric keypad or a reduced set of keys associated with particular functions. Programmer 24 can additionally or alternatively include a peripheral pointing device, such as a mouse, via which a user may interact with the user interface. In some embodiments, a display of programmer 24 may include a touch screen display, and a user may interact with programmer 24 via the display. Through the graphical user interface on programmer 24, a user may select one or more optimized parameters.

Additionally, various pacing settings may be adjusted, or configured, based on various sensed signals. For example, various near-field and far-field signals may be sensed by one or more electrodes of the IMD 16 and/or other devices operatively coupled thereto. For example, Vp to QRS end or offset within a near-field or far-field signal may be used to adjust or configure the AV delay of cardiac conduction system pacing therapy. Further, for example, QRS within a near-field or far-field signal may be used to adjust or configure the VV delay between cardiac conduction system pacing therapy and traditional left ventricular pacing therapy. QRS duration is the time from which the Q wave is detected until the S wave ends.

In one or more embodiments, near-field or far-field signals may be used to determine a surrogate LV activation time, which in turn may be used to determine effective LBB capture and LBB pacing. Surrogate LV activation time may be an activation time as described above, including a cardiac conduction system pacing pulse (e.g., LBB pacing pulse) to breakout ventricular myocardial depolarization (e.g., RV depolarization as measured using electrical signal QRS morphology).

Each of the following illustrative embodiments will be discussed herein in further detail. For example, one or more EGM signals may be monitored during delivery of LBB pacing at a plurality of AV delays or at a plurality of pacing rates, for example, and then analyzed to determine a surrogate LV activation time, which may be used to determined effective LBB capture. Using EGM signals, for example, may advantageously provide more efficient or more effective determination of LBB capture, and may further negate the need for a patient to visit a clinic in person to use ECGs placed on the patient by a medical provider. Although EGM signals are described as being utilized, it is to be understood that any other type of electrical signal known to a person skilled in the art may be monitored.

The electrical activity signals may be acquired from the right ventricle (RV), for example, or anywhere else in or around the heart such as the LBB, left ventricle (LV), etc. For example, a QRS peak within the monitored electrical activity may be used to determine a surrogate LV activation time, and then determine effective LBB capture based on the surrogate LV activation time. Further, for example, a maximum slope prior to a QRS peak within the monitored electrical activity may be used to determine a surrogate LV activation time, and then determine effective LBB capture and pacing based on the surrogate LV activation time.

Still further, near-field and/or far-field electrical signals may be used to determine target pacing settings resulting in specific QRS morphologies, or resulting in specific QRS morphologies over time. For one example, the far-field electrical signals may be sensed in a far-field electrogram (EGM) monitored by IMD 16 and a corresponding lead or a separate device, such as a subcutaneously implanted device.

As used herein, the term “far-field” electrical signal refers to the result of measuring cardiac activity using a sensor, or electrode, positioned outside of an area of interest. For example, an EGM signal measured from an electrode positioned outside of the RV (or the heart chamber of interest) of the patient's heart is one example of a far-field electrical signal of the patient's heart. The sensor, or electrode, may be positioned in an adjacent chamber to the heart chamber of interest.

As used herein, the term “near-field” electrical signal refers to the result of measuring cardiac activity using a sensor, or electrode, positioned near an area of interest. For example, an EGM signal measured from an electrode positioned on the right side of the patient's ventricular septum is one example of a near-field electrical signal of the patient's RV.

R-wave timing is the time in which QRS is detected. Typically, R-wave timing includes using the maximal first derivative of an R-wave upstroke (or the time of the maximal R-wave value). R-wave timing is also used in the device marker channel to indicate the time of the R-wave or the time of ventricular activation.

Pacing-RV sensing or pacing-LV sensing (e.g., pacing-to-RV sensing or pacing-to-LV sensing) is the time interval from the pacing (or pacing artifact) to the time of RV or LV sensing. For example, if pacing-RV sensing is much longer than pacing-LV sensing, this may indicate that the LV activation is occurring much earlier than RV activation (so pacing-RV sensing is longer), then RV pacing may be delivered in synchronization with bundle pacing, so RV and LV activation can occur approximately at the same time.

A user, such as a physician, technician, or other clinician, may interact with programmer 24 to communicate with IMD 16. For example, the user may interact with programmer 24 to retrieve physiological or diagnostic information from IMD 16. One illustrative IMD 16 is described in the Medtronic AMPLIA MRITM CRT-D SURESCAN™ DTMB2D1 manual, which is incorporated by reference in its entirety. A user may also interact with programmer 24 to program IMD 16, e.g., select values for operational parameters of the IMD.

IMD 16 and programmer 24 may communicate via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the IMD 16 implant site in order to improve the quality or security of communication between IMD 16 and programmer 24.

FIG. 3A is a conceptual diagram illustrating IMD 16 and leads 18, 20, 23 of therapy system 10 in greater detail. The triple chamber IMD 16 may be used for cardiac rhythm therapy and defibrillation or cardioversion therapy (CRT-D). Leads 18, 20, 23 may be electrically coupled to a stimulation generator, a sensing module, or other modules of IMD 16 via connector block 34. In some examples, proximal ends of leads 18, 20, 23 may include electrical contacts that electrically couple to respective electrical contacts within connector block 34. In addition, in some examples, leads 18, 20, 23 may be mechanically coupled to connector block 34 with the aid of set screws, connection pins, or another suitable mechanical coupling mechanism.

Each of the leads 18, 20, 23 includes an elongated, insulative lead body, which may carry any number of concentric coiled conductors separated from one another by tubular, insulative sheaths. In the illustrated example, an optional pressure sensor 38 and bipolar electrodes 40 and 42 are located proximate to a distal end of lead 18. In addition, bipolar electrodes 44 and 46 are located proximate to a distal end of lead 20 and bipolar electrodes 48 and 50 are located proximate to a distal end of lead 23. In FIG. 3A, pressure sensor 38 is disposed in right ventricle 28. Pressure sensor 38 may respond to an absolute pressure inside right ventricle 28, and may be, for example, a capacitive or piezoelectric absolute pressure sensor. In other examples, pressure sensor 38 may be positioned within other regions of heart 12 and may monitor pressure within one or more of the other regions of heart 12, or pressure sensor 38 may be positioned elsewhere within or proximate to the cardiovascular system of patient 14 to monitor cardiovascular pressure associated with mechanical contraction of the heart. Optionally, a pressure sensor in the pulmonary artery can be used that is in communication with IMD 16.

Electrodes 40, 44 and 48 may take the form of ring electrodes, and electrodes 42, 46 and 50 may take the form of extendable and/or fixed helix tip electrodes mounted within insulative electrode heads 52, 54 and 56, respectively. Each of electrodes 40, 42, 44, 46, 48 and 50 may be electrically coupled to a respective one of the coiled conductors within the lead body of its associated lead 18, 20, 23, and thereby coupled to respective ones of the electrical contacts on the proximal end of leads 18, 20 23.

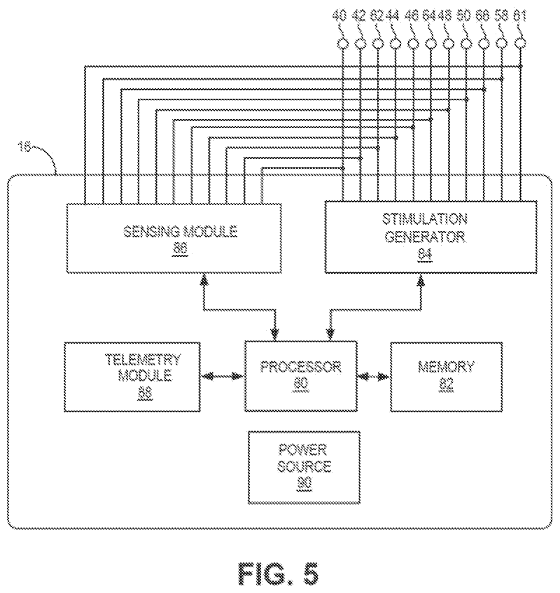

Electrodes 40, 42, 44, 46, 48 and 50 may sense electrical signals attendant to the depolarization and repolarization of heart 12. The electrical signals are conducted to IMD 16 via the respective leads 18, 20, 23. In some examples, IMD 16 also delivers pacing pulses via electrodes 40, 42, 44, 46, 48, 50 to cause depolarization of cardiac tissue of heart 12. In some examples, as illustrated in FIGS. 3A-3B, IMD 16 includes one or more housing electrodes, such as housing electrode 58, which may be formed integrally with an outer surface of hermetically-sealed housing 60 of IMD 16 or otherwise coupled to housing 60. In some examples, housing electrode 58 may be defined by an uninsulated portion of an outward facing portion of housing 60 of IMD 16. Electrode 50 may be used for pacing and/or sensing of the cardiac conduction system tissue (e.g., LBB, His bundle or other bundle branch tissue such as RBB). Other divisions between insulated and uninsulated portions of housing 60 may be employed to define two or more housing electrodes. In some examples, housing electrode 58 includes substantially all of housing 60. Any of the electrodes 40, 42, 44, 46, 48 and 50 may be used for unipolar sensing or pacing in combination with housing electrode 58 or for bipolar sensing with two electrodes in the same pacing lead. In one or more embodiments, housing 60 may enclose a stimulation generator (see FIG. 5) that generates cardiac pacing pulses and defibrillation or cardioversion shocks, as well as a sensing module for monitoring the patient's heart rhythm.

Leads 18, 20, 23 may also include elongated electrodes 62, 64, 66, respectively, which may take the form of a coil. IMD 16 may deliver defibrillation shocks to heart 12 via any combination of elongated electrodes 62, 64, 66, and housing electrode 58. Electrodes 58, 62, 64, 66 may also be used to deliver cardioversion pulses to heart 12. Electrodes 62, 64, 66 may be fabricated from any suitable electrically conductive material, such as, but not limited to, platinum, platinum alloy or other materials known to be usable in implantable defibrillation electrodes.

Pressure sensor 38 may be coupled to one or more coiled conductors within lead 18. In FIG. 3A, pressure sensor 38 is located more distally on lead 18 than elongated electrode 62. In other examples, pressure sensor 38 may be positioned more proximally than elongated electrode 62, rather than distal to electrode 62. Further, pressure sensor 38 may be coupled to another one of the leads 20, 23 in other examples, or to a lead other than leads 18, 20, 23 carrying stimulation and sense electrodes. In addition, in some examples, pressure sensor 38 may be self-contained device that is implanted within heart 12, such as within the septum separating right ventricle 28 from left ventricle 32, or the septum separating right atrium 26 from left atrium 33. In such an example, pressure sensor 38 may wirelessly communicate with IMD 16.

FIG. 3B shows IMD 16 coupled to leads 18, 20, 22, 23. Right atrial (RA) lead 22 may extend through one or more veins and the vena cava, and into the right atrium 26 of heart 12. RA lead 22 may be connected to triple chamber IMD 16, e.g., using a Y-adaptor. IMD 16 may be used for cardiac rhythm therapy and defibrillation or cardioversion therapy (CRT-D). RA lead 22 may include electrodes that are the same or similar to the electrodes of lead 18, 20, 23, such as ring electrodes 40, 44 and 48, extendable helix tip electrodes 42, 46 and 50, and/or elongated electrodes 62, 64, 66, in the form of a coil.

FIGS. 3C-D show patient's heart 12 implanted with IMD 716 operably coupled to implantable medical electrical lead 723 to deliver bundle branch pacing according to one example of an IMD system 710. FIG. 3D is a close-up view of lead 723 in the patient's heart 12 of FIG. 3C. In some embodiments, electrical lead 723 may be the only lead implanted in the patient's heart 12.

In particular, lead 723 may be configured for dual or single bundle branch pacing. Lead 723 may be the same as or similar to lead 23 (FIGS. 2A-B), except lead 723 is implanted near the bundle branches instead of, for example, the His bundle 13. As illustrated, lead 723 is implanted in the septal wall from RV 28 toward LV 32. Lead 723 may not pierce through the wall of LV 32 or extend into the LV chamber. Electrode 752 and tissue-piercing electrode assembly 761 may be disposed on a distal end portion of lead 723, which may also be described as a shaft. Electrode 752 and tissue-piercing electrode assembly 761 may be the same as or similar to electrode and tissue-piercing electrode assembly 50 (FIG. 3A), except electrode 752 is configured as a cathode electrode to sense or pace the RBB and electrode assembly 761 is configured to sense or pace the LBB, for example, during dual bundle branch pacing. Accordingly, electrode 752 may be implanted near RBB 8b, and electrode assembly 761 may be implanted near LBB 8a. During single bundle branch pacing, for example, one of electrode 752 and electrode assembly 761 may be used to pace only the RBB or the LBB, respectively.

Electrode assembly 761 may be described as a unipolar cathode electrode, which may be implanted towards the left side of the patient's septum. Electrode 752 may be described as a unipolar cathode electrode, which may be implanted towards the right side of the patient's septum.

During dual bundle branch pacing, both electrode 752 and electrode assembly 761 (which also includes an electrode) may each deliver a cathodal pulse to achieve synchronized activation, or excitation, of RBB 8b and LBB 8a, which may result in synchronized activation of RV 28 and LV 32. In some embodiments, the pulses may be delivered at the same time to achieve synchrony. In other embodiments, the pulses may be delivered with a delay to achieve synchrony.

Lead 723 may include electrode 770 disposed more proximal to the electrode 752 and electrode assembly 761. Electrode 770 may be positioned in or near RA 26 and may function as an anode for cathodal pulses from electrode 752 and/or electrode assembly 761.

The configuration of therapy system 10 illustrated in FIGS. 2A-4 are merely examples. In other examples, a therapy system may include epicardial leads and/or patch electrodes instead of or in addition to the transvenous leads 18, 20, 22 and/or cardiac conduction system pacing lead 23 illustrated in FIGS. 2A-4 or other configurations shown or described herein or incorporated by reference. Further, IMD 16 need not be implanted within patient 14. In examples in which IMD 16 is not implanted in patient 14, IMD 16 may deliver defibrillation shocks and other therapies to heart 12 via percutaneous leads that extend through the skin of patient 14 to a variety of positions within or outside of heart 12.

In other examples of therapy systems that provide electrical stimulation therapy to heart 12, such therapy systems may include any suitable number of leads coupled to IMD 16, and each of the leads may extend to any location within or proximate to heart 12. For example, therapy systems may include three transvenous leads located as illustrated in FIGS. 2A-4, and an additional lead located within or proximate to left atrium 33 (FIG. 1). As another example, therapy systems may include a single lead that extends from IMD 16 into right atrium 26 or right ventricle 28, or two leads that extend into a respective one of right ventricle 28 and right atrium 26. An example of this type of therapy system is shown in FIGS. 3A-3B. If four leads are required for therapy delivery, an IS-1 connector may be used in conjunction with Y-adaptor 25 extending from the RA port of the connector. The Y-adaptor allows two separate leads—e.g., right atrial lead and the bundle pacing bundle lead—to extend from the two separate legs of the “Y shape” while the single leg is inserted into connector block 34 on IMD 16.

FIG. 4 is a conceptual diagram illustrating another example of therapy system 70. Therapy system 70 shown in FIG. 4 may be useful for providing defibrillation and pacing pulses to heart 12. Therapy system 70 is similar to therapy system 10 of FIGS. 2A-B or 3A-D, but includes two leads 18, 23, rather than three leads. Therapy system 70 may utilize an IMD 16 configured to deliver, or perform, dual chamber pacing. Leads 18, 23 are implanted within right ventricle 28 and right atrium 26 to pace one or more portions of the cardiac conduction system such as the His bundle or one or both bundle branches, respectively.

Cardiac conduction system pacing lead 23 may be in the form of a helix (also referred to as a helical electrode) may be positioned proximate to, near, adjacent to, or in, area or portions of the cardiac conduction system such as, e.g., ventricular septum, triangle of Koch, the His bundle, left bundle branch tissues, and/or right bundle branch tissue. Cardiac conduction system pacing lead 23 may be configured as a bipolar lead or as a quadripolar lead that may be used with a pacemaker device, a CRT-P device or a CRT-ICD.

FIG. 5 is a functional block diagram of one example configuration of IMD 16, which includes processor 80, memory 82, stimulation generator 84 (e.g., electrical pulse generator or signal generating circuit), sensing module 86 (e.g., sensing circuit), telemetry module 88, and power source 90. One or more components of IMD 16, such as processor 80, may be contained within a housing of IMD 16 (e.g., within a housing of a pacemaker). Telemetry module 88, sensing module 86, or both telemetry module 88 and sensing module 86 may be included in a communication interface. Memory 82 includes computer-readable instructions that, when executed by processor 80, cause IMD 16 and processor 80 to perform various functions attributed to IMD 16 and processor 80 herein. Memory 82 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Processor 80 may include any one or more of a microprocessor, a controller, a digital signal processor (DSP), an application specific integrated circuit (ASIC), a field-programmable gate array (FPGA), or equivalent discrete or integrated logic circuitry. In some examples, processor 80 may include multiple components, such as any combination of one or more microprocessors, one or more controllers, one or more DSPs, one or more ASICs, or one or more FPGAS, as well as other discrete or integrated logic circuitry. The functions attributed to processor 80 herein may be embodied as software, firmware, hardware or any combination thereof. Processor 80 controls stimulation generator 84 to deliver stimulation therapy to heart 12 according to a selected one or more of therapy programs (e.g., optimization of the AV delay, VV delay, VV delay etc.), which may be stored in memory 82. Specifically, processor 80 may control stimulation generator 84 to deliver electrical pulses with amplitudes, pulse widths, frequency, or electrode polarities specified by the selected one or more therapy programs.

In some embodiments, RA lead 22 may be operably coupled to electrode 61, which may be used to monitor or pace the RA. Stimulation generator 84 may be electrically coupled to electrodes 40, 42, 44, 46, 48, 50, 58, 61, 62, 64, and 66, e.g., via conductors of respective lead 18, 20, 22, 23 or, in the case of housing electrode 58, via an electrical conductor disposed within housing 60 of IMD 16. Stimulation generator 84 may be configured to generate and deliver electrical stimulation therapy to heart 12. For example, stimulation generator 84 may deliver defibrillation shocks to heart 12 via at least two of electrodes 58, 62, 64, 66. Stimulation generator 84 may deliver pacing pulses via ring electrodes 40, 44, 48 coupled to leads 18, 20, 23, respectively, and/or helical electrodes 42, 46, and 50 of leads 18, 20, or 23, respectively. Cardiac conduction system pacing therapy can be delivered through cardiac conduction system lead 23 that is connected to an atrial, RV, or LV connection port of connector block 34. In some embodiments, the cardiac conduction system pacing therapy can be delivered through leads 18 and/or 23. In some examples, stimulation generator 84 delivers pacing, cardioversion, or defibrillation stimulation in the form of electrical pulses. In other examples, stimulation generator 84 may deliver one or more of these types of stimulation in the form of other signals, such as sine waves, square waves, or other substantially continuous time signals.

Stimulation generator 84 may include a switch module and processor 80 may use the switch module to select, e.g., via a data/address bus, which of the available electrodes are used to deliver defibrillation shocks or pacing pulses. The switch module may include a switch array, switch matrix, multiplexer, or any other type of switching device suitable to selectively couple stimulation energy to selected electrodes.

Sensing module 86 monitors signals from at least one of electrodes 40, 42, 44, 46, 48, 50, 58, 61, 62, 64 or 66 in order to monitor electrical activity of heart 12, e.g., via electrical signals, such as electrocardiogram (ECG) signals and/or electrograms (EGMs). Sensing module 86 may also include a switch module to select which of the available electrodes are used to sense the heart activity. In some examples, processor 80 may select the electrodes that function as sense electrodes via the switch module within sensing module 86, e.g., by providing signals via a data/address bus. In some examples, sensing module 86 includes one or more sensing channels, each of which may include an amplifier. In response to the signals from processor 80, the switch module may couple the outputs from the selected electrodes to one of the sensing channels.

In some examples, one channel of sensing module 86 may include an R-wave amplifier that receives signals from electrodes 44, 46, which are used for pacing and sensing proximate to left ventricle 32 of heart 12. Another channel may include another R-wave amplifier that receives signals from electrodes 40, 42, which are used for pacing and sensing in right ventricle 28 of heart 12. In some examples, the R-wave amplifiers may take the form of an automatic gain-controlled amplifier that provides an adjustable sensing threshold as a function of the measured R-wave amplitude of the heart rhythm.

In addition, in some examples, one channel of sensing module 86 may include a P-wave amplifier that receives signals from electrodes 48, 50, which are used for pacing and sensing in right atrium 26 of heart 12. In some examples, the P-wave amplifier may take the form of an automatic gain-controlled amplifier that provides an adjustable sensing threshold as a function of the measured P-wave amplitude of the heart rhythm. Examples of R-wave and P-wave amplifiers are described in U.S. Pat. No. 5,117,824 to Keimel et al., which issued on Jun. 2, 1992 and is entitled, “APPARATUS FOR MONITORING ELECTRICAL PHYSIOLOGIC SIGNALS,” and is incorporated herein by reference in its entirety. Other amplifiers may also be used. Furthermore, in some examples, one or more of the sensing channels of sensing module 86 may be selectively coupled to housing electrode 58, or elongated electrodes 62, 64, or 66, with or instead of one or more of electrodes 40, 42, 44, 46, 48 or 50, e.g., for unipolar sensing of R-waves or P-waves in any of chambers 26, 28, or 32 of heart 12.

In some examples, sensing module 86 includes a channel that includes an amplifier with a relatively wider pass band than the R-wave or P-wave amplifiers or a high-resolution amplifier with relatively narrow-pass band for His bundle or bundle branch potential recording. Signals from the selected sensing electrodes that are selected for coupling to this wide-band amplifier may be provided to a multiplexer, and thereafter converted to multi-bit digital signals by an analog-to-digital converter for storage in memory 82 as an electrogram (EGM). In some examples, the storage of such EGMs in memory 82 may be under the control of a direct memory access circuit. Processor 80 may employ digital signal analysis techniques to characterize the digitized signals stored in memory 82 to detect and classify the patient's heart rhythm from the electrical signals. Processor 80 may detect and classify the heart rhythm of patient 14 by employing any of the numerous signal processing methodologies known in the art.

If IMD 16 is configured to generate and deliver pacing pulses to heart 12, processor 80 may include pacer timing and control module, which may be embodied as hardware, firmware, software, or any combination thereof. The pacer timing and control module may include a dedicated hardware circuit, such as an ASIC, separate from other processor 80 components, such as a microprocessor, or a software module executed by a component of processor 80, which may be a microprocessor or ASIC. The pacer timing and control module may include programmable counters which control the basic time intervals associated with DDD, VVI, DVI, VDD, AAI, DDI, DDDR, VVIR, DVIR, VDDR, AAIR, DDIR and other modes of single and dual chamber pacing. In the aforementioned pacing modes, “D” may indicate dual chamber, “V” may indicate a ventricle, “I” may indicate inhibited pacing (e.g., no pacing), and “A” may indicate an atrium. The first letter in the pacing mode may indicate the chamber that is paced, the second letter may indicate the chamber in which an electrical signal is sensed, and the third letter may indicate the chamber in which the response to sensing is provided.

Intervals defined by the pacer timing and control module may include atrial and ventricular pacing escape intervals, refractory periods during which sensed P-waves and R-waves are ineffective to restart timing of the escape intervals, and the pulse widths of the pacing pulses. As another example, the pace timing and control module may define a blanking time period and provide signals from sensing module 86 to blank one or more channels, e.g., amplifiers, for a period during and after delivery of electrical stimulation to heart 12. The durations of these intervals may be determined by processor 80 in response to stored data in memory 82. The pacer timing and control module may also determine the amplitude of the cardiac pacing pulses.

During pacing, escape interval counters within the pacer timing/control module may be reset upon sensing of R-waves and P-waves. Stimulation generator 84 may include pacer output circuits that are coupled, e.g., selectively by a switching module, to any combination of electrodes 40, 42, 44, 46, 48, 50, 58, 61, 62, or 66 appropriate for delivery of a bipolar or unipolar pacing pulse to one of the chambers of heart 12. Processor 80 may reset the escape interval counters upon the generation of pacing pulses by stimulation generator 84, and thereby control the basic timing of cardiac pacing functions, including anti-tachyarrhythmia pacing.

In some examples, processor 80 may operate as an interrupt driven device, and is responsive to interrupts from pacer timing and control module, where the interrupts may correspond to the occurrences of sensed P-waves and R-waves and the generation of cardiac pacing pulses. Any necessary mathematical calculations to be performed by processor 80 and any updating of the values or intervals controlled by the pacer timing and control module of processor 80 may take place following such interrupts. A portion of memory 82 may be configured as a plurality of recirculating buffers, capable of holding series of measured intervals, which may be analyzed by processor 80 in response to the occurrence of a pace or sense interrupt to determine whether the patient's heart 12 is presently exhibiting atrial or ventricular tachyarrhythmia.

Telemetry module 88 includes any suitable hardware, firmware, software or any combination thereof for communicating with another device, such as programmer 24 (FIG. 2A). Under the control of processor 80, telemetry module 88 may receive downlink telemetry from and send uplink telemetry to programmer 24 with the aid of an antenna, which may be internal and/or external. Processor 80 may provide the data to be uplinked to programmer 24 and the control signals for the telemetry circuit within telemetry module 88, e.g., via an address/data bus. In some examples, telemetry module 88 may provide received data to processor 80 via a multiplexer.

The various components of IMD 16 are coupled to power source 90, which may include a rechargeable or non-rechargeable battery. A non-rechargeable battery may be selected to last for several years, while a rechargeable battery may be inductively charged from an external device, e.g., on a daily or weekly basis.

The illustrative devices and methods described herein may provide and use monitored electrical activity (e.g., EGM, etc.) to determine effective LBB capture to provide effective LBB pacing for use in cardiac therapy such as, for example, cardiac resynchronization therapy (CRT), biventricular pacing, etc. Further, the monitored electrical activity may be used to determine a surrogate LV activation time, which may in turn be used to determine effective LBB capture. As will be described further herein, electrical activity of the heart may be monitored for specific areas of the heart, such as a specific chamber (e.g., RV, LV), and may be acquired using various devices as described herein (e.g., EGM).

The surrogate LV activation time may be determined, for example, by analyzing the monitored electrical activity. In one embodiment, the monitored electrical activity may include one or more EGM signal(s) as monitored from the RV using an RV tip electrode as described herein. The surrogate LV activation time may be determined, or measured, from the delivery of a pacing pulse to the cardiac conduction system (e.g., the LBB) to a fiducial point within the one or more EGM signals. For example, the surrogate LV activation time may be determined, or measured, from the delivery of a pulse to the cardiac conduction system (e.g., the LBB) to a QRS peak in the EGM signal. Further, for example, the surrogate LV activation time may be determined, or measured, from delivery of the pacing pulse to the cardiac conduction system (e.g., the LBB) to a signal maximum slope prior to QRS peak in the EGM signal. It is to be understood that any other EGM signal parameter may be used. The determined surrogate LV activation time, in any case, may be used to determine effective LBB capture and effective LBB pacing. For example, the EGM signal parameter, and the resulting determined surrogate LV activation time, may be monitored, and determined, at different paced settings. If the surrogate LV activation time remains consistent at the different paced settings, this may indicate effective capture of the LBB. In another embodiment, if the surrogate LV activation time remains consistent with a template LV activation time, this may indicate effective capture of the LBB.

IMD utilize the surrogate LV activation time to determine whether effective capture of the LBB is occurring and to adjust one or more pacing parameters until effective LBB capture is achieved. Additionally, the surrogate LV activation time may be monitored over time to ensure that continuing cardiac conduction system pacing is capturing the LBB. Illustrative pacing therapy may be delivered using the devices as described in FIGS. 2-5.

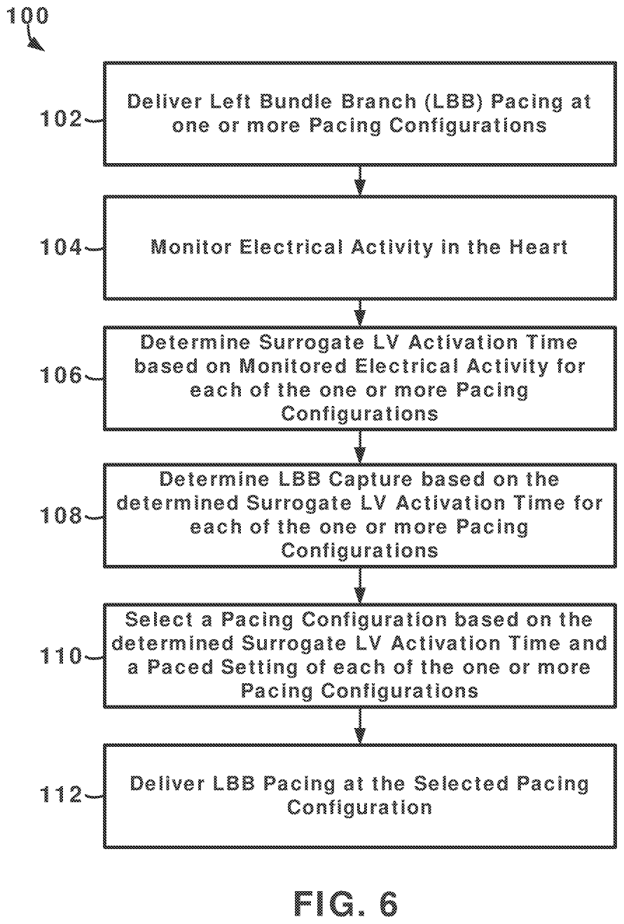

An illustrative method 100 of determining effective LBB capture and LBB pacing based on monitored electrical activity that may be utilized by the devices of FIGS. 2-5 is depicted in FIG. 6. The method 100 may include delivering LBB pacing at one or more pacing configurations 102. Various embodiments describing delivery of cardiac conduction system pacing are described above (e.g., using an IMD). When a cardiac conduction system pacing pulse is initiated, it may be described as being conducted or propagated very quickly through the cardiac conduction system, and then conducted or propagated more slowly through the ventricular myocardium before breakout myocardial depolarization occurs.

In some embodiments where the cardiac conduction system pulse is delivered to the left bundle branch, the pacing pulse is conducted from the left bundle branch (LBB) to breakout right ventricle (RV) myocardial depolarization. In such embodiments, the LBB also needs to be effectively captured such that a large portion of the breakout RV myocardial depolarization occurs via the activation of the LBB (as opposed to, for example, myocardial activation near an implanted electrode).

The one or more pacing configurations may include one or more of pacing output voltage delivered to the LBB, number of pacing pulses, position of pacing electrode, timing between pacing pulses (otherwise known as the paced rate, or heart rate), atrio-ventricular (AV) delay, inter-ventricular (VV) delay, pacing polarity, pacing pulse width, etc. Different pacing configurations may provide pacing therapy (e.g., CRT) that effectively captures the LBB, which may result in improved stroke volume and cardiac output.

In response to or during delivery of LBB pacing at one or more pacing configurations 102, the method 100 may further include monitoring electrical activity in the heart 104. Monitoring electrical activity in the heart may include monitoring near-field electrical signals or far-field electrical signals using the systems and devices as described above. Monitoring electrical activity may be accomplished using the sensing module 86, for example, as described above. Electrical activity may include a near-or far-field electrogram (EGM) signal as sensed from the at least one implanted electrode. In one or more embodiments, electrical activity may be monitored in the right ventricle (RV) of the heart. In alternate embodiments, electrical activity may be monitored in another chamber or elsewhere in, on, or around the heart, such as the LBB, left or right ventricular septum, etc. The sensing module 86 or other sensing apparatus may monitor the electrical activity (e.g., EGM signal) during the delivery of the LBB pacing 102.

In one embodiment, monitoring electrical activity may include use of one or more implanted electrodes as discussed above with respect to FIGS. 1-5, such as a tip electrode to an RV coil electrode unipolar EGM, a tip electrode to a housing electrode unipolar EGM, an RV coil electrode to a housing electrode unipolar EGM, and an atrial ring electrode to a housing electrode unipolar EGM. A tip electrode may be an electrode located on the tip of any lead as described above. A coil electrode may be an electrode shaped in or on a coil and located proximal to the tip of the lead along the lead body. A ring electrode may be an electrode shaped in a ring around the lead body and located proximal to the tip of the lead along the lead body. A housing electrode may be an electrode located in or about the housing of the IMD. Location identifiers such as RV are examples of possible electrode locations. For example, the “atrial” location may be one of either atrium. In alternative embodiments, bipolar EGM signals may be monitored.

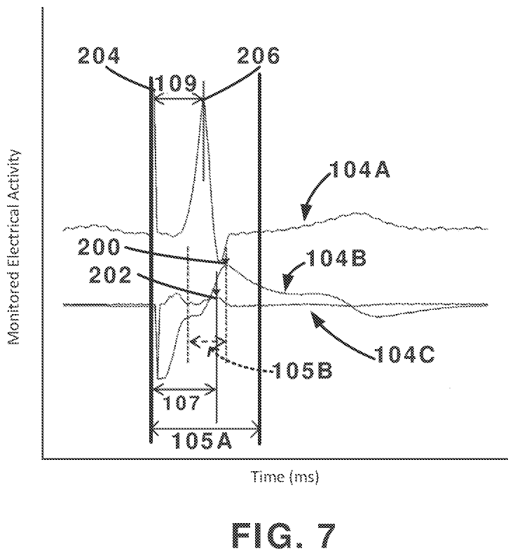

In one embodiment, and as described above, an RV unipolar electrode (e.g., RV tip to can unipolar implanted electrode) may be used to monitor electrical activity of the RV. The RV unipolar electrode may produce an EGM signal 104B, as illustrated and labeled in FIG. 7, which also includes an exemplary ECG 104A over the same time period, illustrating the monitored electrical activity of the method 100 of FIG. 6. More specifically, FIG. 7 illustrates an ECG signal 104A, which may be produced using one or more surface electrodes (e.g., a standard 12-lead ECG), including electrodes positioned on the surface of a patient near or at the standard I, II, III, IV, V, and VI chest leads and/or upper right and left arm and lower right and left leg limb leads. More specifically, ECG signal 104A may be an ECG signal obtained from a V5 (referred to as V above) standard surface electrode lead using a standard 12-lead ECG.

In one embodiment, the EGM signal 104B may be any EGM signal obtained from any electrode. In another embodiment, the EGM signal 104B may be an averaged EGM signal obtained from more than one electrode. In another embodiment, the EGM signal 104B is obtained from the RV unipolar electrode. FIG. 7 also illustrates and labels a signal dedicated as 104C, which corresponds to a calculated differential signal based on the EGM signal 104B. A differential signal illustrates the rates of change over time of the original signal.

The ECG signal 104A is used to illustrate LV activation time (“LVAT”) 109 on a typical QRS waveform. Various electrodes as discussed herein may be used to define LVAT 109. The LVAT 109 is the time interval from delivery of a pacing pulse (corresponding to a pacing spike on the ECG signal 104A) to the R-wave peak. As shown, the LVAT 109 is identified in the ECG signal 104A from the delivery of the pacing pulse 204 to the R-wave peak 206.

The method 100 may further include determining a surrogate LV activation time 107 based on the monitored electrical activity for each of the one or more pacing configurations 106. Surrogate LV activation time is representative of LVAT. In one embodiment, the surrogate LV activation time 107 is illustrated using the EGM signal 104B. In another embodiment, the surrogate LV activation time 107 is illustrated using the differential EGM signal 104C. For example, the surrogate LV activation time 107 extends from delivery of a pacing pulse to a fiducial point on the EGM 104B or on the differential EGM 104C signals, as discussed below. The fiducial point discussed herein may be any measurable point within the far-field electrical activity signal or derivatives thereof (e.g., the differential signal) that corresponds, or correlates, to the actual LVAT. To determine the fiducial point, the devices and systems as described above may identify the fiducial point using any calculations or methodologies, such as by using sensing module 86 as described herein, processor 80 as described herein, etc. Some examples of fiducial points may include one or more of the following: onset of a QRS morphology or any specific points therein, maximum signal amplitude, minimum signal amplitude, minimum and maximum signal slopes, the point where the monitored electrical activity crosses the isoelectric line (which may be located, for example, at 0 Volts), any other monitored electrical activity signal waveform, etc. In one embodiment, the fiducial point is a QRS peak 200. In another embodiment, the fiducial point is the maximum EGM signal slope (or the peak of the differential EGM signal) 202 in a lookback time window 105B prior to the QRS peak 200, as discussed below.

In one embodiment, the QRS peak 200 may be identified within a first time window 105A extending from delivery of a pacing pulse to a first time point on the EGM signal. The first time window 105A may be greater than or equal to 350 ms, greater than or equal to 400 ms, greater than or equal to 450 ms, greater than or equal to 500 ms, greater than or equal to 550 ms, greater than or equal to 600 ms, etc. and/or less than or equal to 350 ms, less than or equal to 300 ms, less than or equal to 250 ms, less than or equal to 200 ms, less than or equal to 150 ms, less than or equal to 100 ms, etc. The first time window 105A may be any first time window within a selected range, as shown in FIG. 7. In another embodiment, no time window may be used, and instead the first QRS peak identified after a pacing pulse is delivered may be used as the fiducial point. The QRS peak 200 may be identified in various ways, as described above.

In one embodiment, as described herein, a lookback time window 105B may be used to identify the fiducial point of the maximum EGM signal slope 202 (prior to the QRS peak 200). The lookback time window 105B may be greater than or equal to 60 ms, greater than or equal to 65 ms, greater than or equal to 70 ms, greater than or equal to 75 ms, greater than or equal to 80 ms, greater than or equal to 100 ms, etc. and/or less than or equal to 60 ms, less than or equal to 55 ms, less than or equal to 50 ms, less than or equal to 45 ms, less than or equal to 40 ms, less than or equal to 35 ms, etc. The lookback time window 105B may be any lookback time window within a selected range, as shown in FIG. 7. In another embodiment, no lookback time window may be used, and instead the first maximum EGM signal slope 202 identified prior to the initial QRS peak after a pacing pulse is delivered may be used as the fiducial point. The maximum EGM slope 202 may be identified in various ways, as described above.

The determined surrogate LV activation time 107 value may be the time interval from delivery of a pacing pulse to the fiducial point. As illustrated in FIG. 7, in one embodiment, the fiducial point is the maximum EGM signal slope 202.

The method 100 may further include determining LBB capture based on the determined surrogate LV activation time 107 for each of the one or more pacing configurations 108. In one embodiment, determining LBB capture may be based on the determined surrogate LV activation time 107 remaining relatively consistent for each of the one or more pacing configurations. Similar to determining LBB capture based on typical ECG and typical LV activation time remaining constant and remaining short, as discussed herein, the EGM signal and the surrogate LV activation 107 time may be used to determine LBB capture.

The relative consistency of the surrogate LV activation times 107 at the one or more pacing configurations may be any chosen or set defined consistency. For example, the consistency may be defined as having the surrogate LV activation time 107 at one pacing configuration (of the one or more pacing configurations) remain within about 9% of the surrogate LV activation time 107 at a prior pacing configuration (e.g., the pacing configuration used just prior, or another pacing configuration used before that). In other embodiments, the consistency as defined may remain within about 5%, or about 10%, or about 15%, or about 20%, or about 25%, or about 30%, or about 35%, or about 40%, or about 50%, or about 60%, or about 70%, or about 80%, or about 90% of the surrogate LV activation time 107 at the prior pacing configuration. In another embodiment, the consistency may be defined in terms of a ratio as opposed to a percentage or may be defined in terms of a set surrogate LV activation time value, or threshold value. In another embodiment, the consistency may be any consistency within a selected range.

In another embodiment, the relative consistency of the surrogate LV activation times 107 at the one or more pacing configurations may be defined as having the surrogate LV activation time 107 at one paced heartbeat at one pacing configuration within about 9% of the surrogate LV activation time 107 at a prior paced heartbeat at the same pacing configuration (e.g., the heartbeat directly prior, or another prior heartbeat). In other embodiments, the consistency as defined may remain within about 5%, or about 10%, or about 15%, or about 20%, or about 25%, or about 30%, or about 35%, or about 40%, or about 50%, or about 60%, or about 70%, or about 80%, or about 90% of the surrogate LV activation time 107 at a prior paced heartbeat. In another embodiment, the consistency may be defined in terms of a ratio as opposed to a percentage, or may be defined in terms of a set surrogate LV activation time value, or threshold value. Still further, for example, the consistency may be any consistency within a selected range.

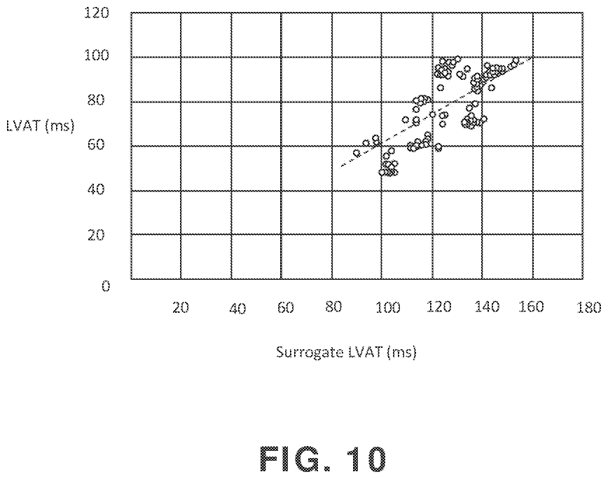

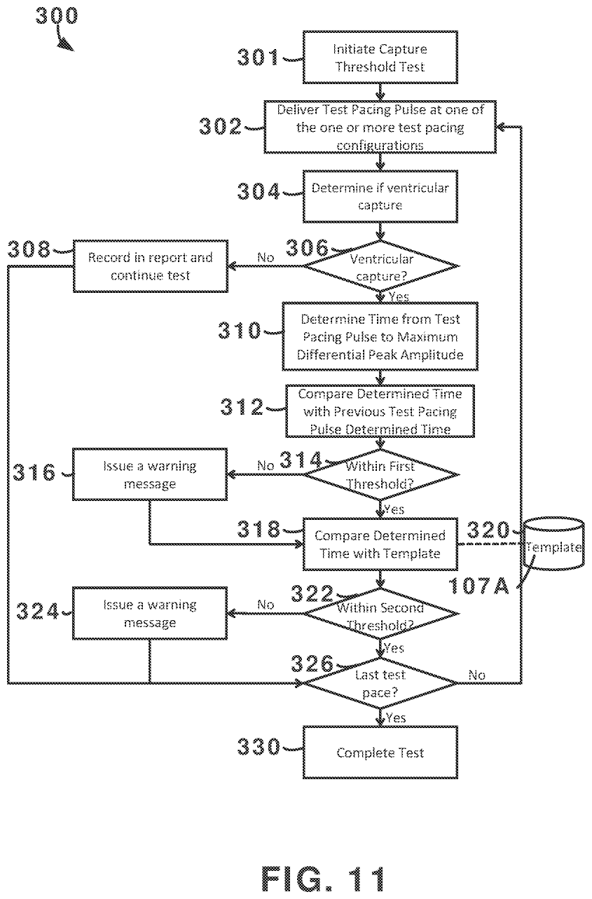

In another embodiment, the surrogate LV activation time 107 may be compared to a template, or threshold, surrogate LV activation time 107A (illustrated in FIGS. 10 and 11). The threshold surrogate LV activation time may be determined from electrical activity of a template paced heartbeat, for example. The comparison of the surrogate LV activation time 107 and the threshold surrogate LV activation time 107A may indicate effective capture of the LBB if the surrogate LV activation time 107 is within about 9% of the threshold surrogate LV activation time 107A. In other embodiments, the consistency as defined may remain within about 5%, or about 10%, or about 15%, or about 20%, or about 25%, or about 30%, or about 35%, or about 40%, or about 50%, or about 60%, or about 70%, or about 80%, or about 90% of the threshold surrogate LV activation time 107A, for example. In another embodiment, the comparison may be defined in terms of a ratio as opposed to a percentage. The consistency may be any consistency within a selected range, for example. In further embodiments, effective capture of the LBB may be determined using any devices, systems, or methods known to a person of ordinary skill in the art.

After determining LBB capture for one or more pacing configurations 108, the method 100 may further include selecting a pacing configuration based on the determined surrogate LV activation time 110. In one embodiment, where the determined surrogate LV activation time 107 is used to determine LBB capture at one of the one or more pacing configurations, such pacing configuration resulting in LBB capture may be beneficial to use as the selected pacing configuration for that patient, and thus, selected. Further, where various determined surrogate LV activation times 107 result in effective LBB capture at more than one of the one or more pacing configurations, then one of the pacing configurations may be selected to use as the selected pacing configuration for the patient based on various criteria. For example, a lower pacing output may result in less energy usage and longer device (e.g., using an IMD) battery life, and thus, if more than one pacing configuration result in the effective LBB capture, then the pacing configuration which uses less energy and results in longer device batter life may be selected.

The method 100 may further include delivering LBB pacing at the selected pacing configuration 112. Various embodiments describing delivery of cardiac conduction system pacing are described above (e.g., using an IMD). Additionally, the method 100 may be performed more than once, so that a medical provider or user may periodically recheck effective LBB capture, change pacing configuration settings, monitor effective capture of the LBB, etc., based on the surrogate LV activation time(s) 107.

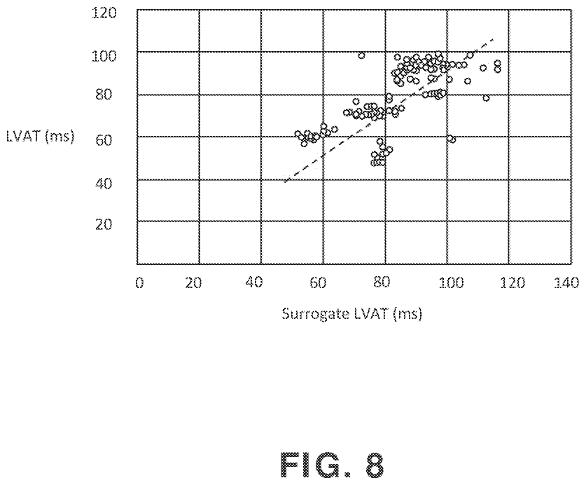

FIG. 8 is a scatter plot illustrating correlation between determined surrogate LV activation times 107 using the method of FIG. 6 and actual LVAT 109. The data illustrated was captured from eight patients at different pacing outputs, and each data point illustrated indicates a representative heartbeat from one patient at one pacing output. Example pacing outputs varied from about 0.5 volts to about 2 volts per pacing pulse. In one or more embodiments, pacing outputs may be less than or equal to 0.5 V, 1V, 1.25V, 1.5V, 1.75V, 2V, 3V, 4V, 5V, 6V, etc. Pacing outputs may be greater than or equal to 0.5V, 1V, 2V, 2.25V, 2.5V, 3V, 4V, 5V, 6V, etc. More specifically, FIG. 8 illustrates the correlation between the surrogate LV activation time 107 based on the time interval from delivery of a pacing pulse corresponding to the fiducial point maximum EGM signal slope 202, and LVAT 109. The LVATs 109 may be correlated to the surrogate LV activation times 107 shown in FIG. 8 using the below formula, and results in an R² value >0.95. As a result, there is high correlation between traditional LVAT 109 and the surrogate LV activation time 107 based on the fiducial point 202, indicating that the surrogate LV activation time 107 may be clinically used similar to traditional LVAT 109.

LVAT=(0.92)*(surrogate LV activation time based on 202)

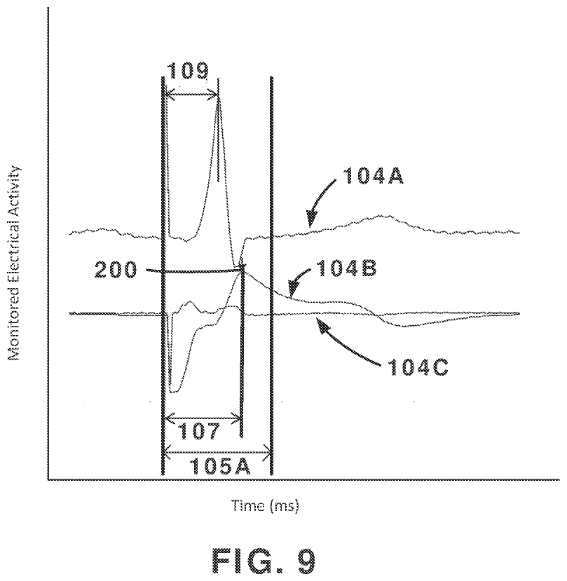

FIG. 9 is another depiction of exemplary ECG signal 104A and exemplary EGM signal 104B and exemplary differential EGM signal 104C over time, illustrating the monitored electrical activity of the method 100 of FIG. 6 according to one or more embodiments. FIG. 9 illustrates the same signals as described above with respect to FIG. 7, and also illustrates the same exemplary LVAT 109. FIG. 9 also illustrates the same QRS peak 200 as a possible fiducial point as is depicted in FIG. 7. The first time window 105A may be the same as described above with respect to FIG. 7. Conversely, FIG. 9 illustrates a surrogate LV activation time 107 based on a time interval that extends from delivery of a pacing pulse to the fiducial point QRS peak 200 (as opposed to the fiducial point of the maximum EGM signal slope 202, as discussed above and as illustrated in FIG. 7). The determined surrogate LV activation time 107 value may be the time interval from delivery of a pacing pulse to the fiducial point. As illustrated in FIG. 9, in one embodiment, the fiducial point is the maximum EGM QRS peak 200.

FIG. 10 is a scatter plot illustrating the correlation between another, different determined surrogate LV activation times 107 of the method 100 of FIG. 6 and actual LVAT 109. The data illustrated was captured from eight patients at different pacing outputs as described above with respect to FIG. 8, and each data point illustrated indicates a representative heartbeat from one patient at one pacing output. More specifically, FIG. 10 illustrates the correlation between the surrogate LV activation time 107 based on the time interval from delivery of a pacing pulse to the fiducial point QRS peak 200, and LVAT 109. The LVATs 109 may be correlated to the surrogate LV activation times 107 using the below formula, and results in an R² value >0.95. As a result, there is high correlation between traditional LVAT 109 and the surrogate LV activation time 107 based on the fiducial point 200, indicating that the surrogate LV activation time 107 may be clinically used similar to traditional LVAT 109.

LVAT=(0.62)*(surrogate LV activation time based on 200)

FIG. 11 is a block diagram of another illustrative method 300 of determining LBB capture using an EGM signal that may be utilized by the devices of FIGS. 2-5 and using one or more processes that are the same or similar to the methods illustrated in FIGS. 6, 7, and 9. The method 300 of FIG. 11 may be applied during capture threshold tests at implant and follow-ups with a programmer. Capture threshold tests may be used to determine the lowest pacing output voltage that still results in effective capture of LBB and effective pacing of LBB (as opposed to, for example, ineffective capture or pacing of the LBB and instead capturing or pacing the ventricular myocardium, such as LV septal pacing, etc.). Such capture threshold test method may include the steps of initiating the capture threshold test 301, delivering a test pacing pulse at one of the one or more test pacing configurations 302, and determining if there is loss of ventricular capture (combined steps 304 and 306 as illustrated in FIG. 11). In response to a determining that there is loss of ventricular capture, the capture threshold test method may further include recording such determination that there is not ventricular capture in a report and continuing the test 308.

In response to determining that there is still ventricular capture, the capture threshold test method may further include determining the time interval from the test pacing pulse to the maximum EGM signal slope 202 (designated as “maximum differential peak amplitude” in FIGS. 11), 310. The determined time interval may instead be the time interval from the test pacing pulse to the QRS peak 200, as described above. The method may further include comparing the determined time with a previous test pacing pulse determined time 312. The method may further include determining if the comparison is within a first threshold 314. In response to a determination that the comparison is not within the first threshold, such determination correlates with non-effective LBB capture, and the method may further include issuing a warning message 316. In response to a determination that the comparison is within the first threshold, such determination correlates with effective LBB capture, and the method may further include comparing the determined time with a template time 318 (designated with the dashed line and the “template” designation 320). The template 320 may be equivalent to the template threshold 107A discussed above.

The method may further include determining that the threshold comparison is within a second threshold 322. In response to a determination that the comparison is not within the second threshold, such determination correlates with non-effective LBB capture, and the method may further include issuing warning message 324. In response to a determination that the comparison is within the second threshold, such determination correlates with effective LBB capture, and the method may further include determining if the last test pace has been delivered 326. In response to a determination that the last test pace has not been delivered, the method may return to the step of delivering a test pacing pulse at one of the one or more test pacing configurations 302. In response to a determination that the last test pace has been delivered, the method may complete the capture threshold test 330. Once the method is complete, the conventional capture threshold may be recorded, reported, etc.

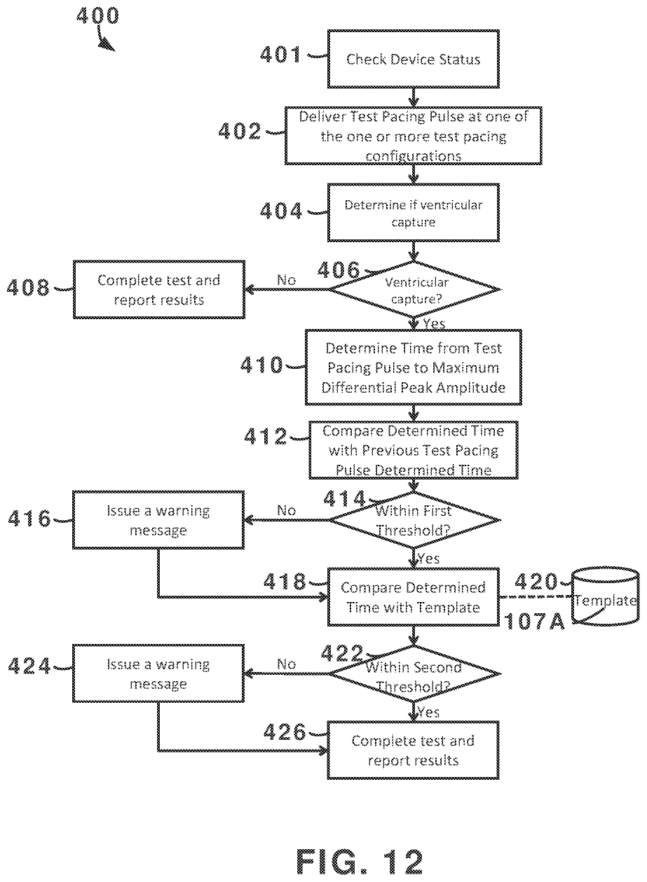

FIG. 12 is a block diagram of another illustrative method 400 of determining LBB capture using an EGM signal that may be utilized by the devices of FIGS. 2-5 and using one or more processes that are the same or similar to the methods illustrated in FIGS. 6, 7, 9, and 11. The method 400 of FIG. 12 may be applied to or integrated with a capture management feature as described herein, and further may be used to chronically track the trend of EGM-based surrogate LV activation time 107 as a monitoring feature over time.

The method 400 of FIG. 12 is similar to the method 300 of FIG. 11 but does not include repetition at various pacing configurations. Instead, the method 400 of FIG. 12 is not repeated and is only performed for one test pacing pulse at one pacing configuration. The results of the method 400 of FIG. 12 may indicate that a patient's LBB is or is not effectively captured, based on the patient's surrogate LV activation time 107. If the results indicate that the patient's LBB is not effectively captured, the change in the patient's surrogate LV activation time 107 may be monitored over time, and in some cases the surrogate LV activation time first and second thresholds (as described below) may be changed or updated.

Method 400 may include the steps of checking device status 401, delivering a test pacing pulse at one of the one or more test pacing configurations 402, and determining if there is loss of ventricular capture (combined steps 404 and 406 as illustrated in FIG. 12). In response to a determining that there is loss of ventricular capture, the capture threshold test method may further include completing the test and reporting the results 408.

In response to determining that there is still ventricular capture, the capture threshold test method may further include determining the time interval from the test pacing pulse to the maximum EGM signal slope 202 (designated as “maximum differential peak amplitude” in FIGS. 12), 410. The determined time interval may instead be the time interval from the test pacing pulse to the QRS peak 200, as described above. The method may further include comparing the determined time with a previous test pacing pulse determined time 412. The method may further include determining if the comparison is within a first threshold 414. In response to a determination that the comparison is not within the first threshold, such determination correlates with non-effective LBB capture, and the method may further include issuing a warning message 416. In response to a determination that the comparison is within the first threshold, such determination correlates with effective LBB capture, and the method may further include comparing the determined time with a template time 418 (designated with the dashed line and the “template” designation 420). The template 420 may be equivalent to the template threshold 107A discussed above.

The method may further include determining that the threshold comparison is within a second threshold 422. In response to a determination that the comparison is not within the second threshold, such determination correlates with non-effective LBB capture, and the method may further include issuing warning message 424. In response to a determination that the comparison is within the second threshold, such determination correlates with effective LBB capture, and the method may complete the test 426. Once the method is complete, the surrogate LV activation time 107, or effective capture of the patient's LBB, may be recorded, reported, etc.

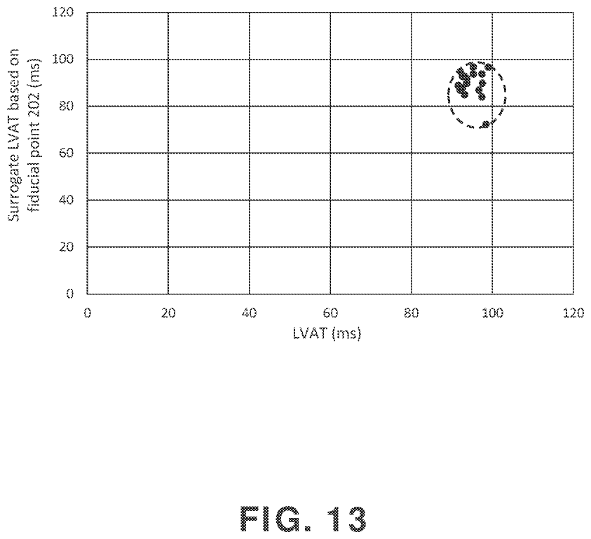

FIG. 13 is a scatter plot illustrating correlation between determined surrogate LV activation times 107 using the method of FIG. 6 and fiducial point 202, and actual LVATs 109. The data illustrated was captured from one patient and at various pacing outputs. Example pacing outputs varied from about 0.75 volts to about 5 volts per pacing pulse. In one or more embodiments, the pacing outputs may be as described herein with respect to FIG. 8. More specifically, FIG. 13 illustrates the correlation between the surrogate LV activation time 107 based on the time interval from delivery of a pacing pulse corresponding to the fiducial point maximum EGM signal slope 202, and LVAT 109, at the different pacing outputs. The aggregated data all appear to be similar, as illustrated with the dotted oval around the aggregated data. Such similarity at various pacing outputs indicates that the surrogate LV activation times 107 did not vary with pacing output. This indication, in turn, indicates that the LBB is effectively captured, because the surrogate LV activation times 107 and LVATs 109 remain constant at various pacing outputs.

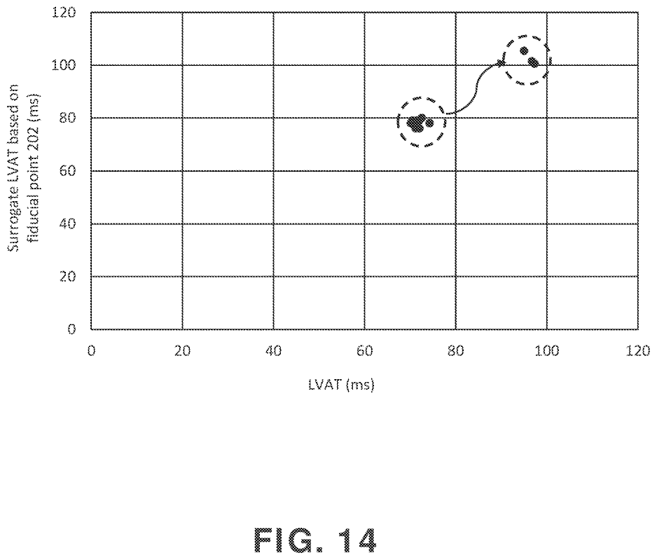

FIG. 14 is a scatter plot illustrating the correlation between determined surrogate LV activation times 107 using the method of FIG. 6 and fiducial point 202, and actual LVATs 109, similar to FIG. 13, but was captured from another, different patient and at various pacing outputs as described above with respect to FIG. 13. In one or more embodiments, the pacing outputs may be as described herein with respect to FIG. 8. As illustrated in FIG. 14, the data do not all appear to be similar, as illustrated with the dotted circles around different groups of the aggregated data. Such difference at various pacing outputs indicates that the surrogate LV activation times 107 did vary with pacing output. This indication, in turn, indicates that the LBB is not effectively captured at least at some of the pacing outputs, because the surrogate LV activation times 107 and LVATs 109 do not remain constant at various pacing outputs. For example, a prolonged LVAT 109, or a prolonged surrogate LV activation time 107, may be indicative of undesirable LV septal pacing and ineffective LBB capture. In one or more embodiments, such aggregate data as illustrated in FIGS. 13 and 14 may be useful to medical providers as another method of determining effective capture of LBB in a patient.

Various examples have been described. These and other examples are within the scope of the following claims. For example, a single chamber, dual chamber, or triple chamber pacemakers (e.g., CRT-P) or ICDs (e.g., CRT-D) devices can be used to implement the illustrative methods described herein.

### ILLUSTRATIVE EXAMPLES

While the present disclosure is not so limited, an appreciation of various aspects of the disclosure will be gained through a discussion of the specific illustrative examples provided below. Various modifications of the illustrative examples, as well as additional examples of the disclosure, will become apparent herein.

Example Ex1: an implantable medical device comprising:
one or more implantable electrodes comprising an LBB electrode positionable proximate a portion of a patient's left bundle branch (LBB); and
a computing apparatus comprising processing circuitry and operably coupled to the one or more implantable electrodes, wherein the computing apparatus is configured to:
deliver LBB pacing proximate to the LBB using the LBB electrode at one or more pacing configurations;
monitor electrical activity using the one or more implantable electrodes during delivery of the LBB pacing at the one or more pacing configurations;
determine a surrogate LV activation time based on the monitored electrical activity for each of the one or more pacing configurations, wherein the surrogate LV activation time is representative of an LV activation time; and
determine whether the LBB pacing has captured the LBB for each of the one or more pacing configurations based on the determined surrogate LV activation time for each of the one or more pacing configurations.

Example Ex2: a method comprising:
delivering LBB pacing proximate to a patient's left bundle branch (LBB) using an LBB electrode positioned proximate a portion of the LBB at one or more pacing configurations;
monitoring electrical activity using one or more electrodes during delivery of the LBB pacing at the one or more pacing configurations;
determining a surrogate LV activation time based on the monitored electrical activity for each of the one or more pacing configurations, wherein the surrogate LV activation time is representative of an LV activation time; and
determining whether the LBB pacing has captured the LBB for each of the one or more pacing configurations based on the determined surrogate LV activation time for each of the one or more pacing configurations.

Example Ex3: the implantable medical device as in Example Ex1 or the method as in Example Ex2, wherein the one or more implantable electrodes further comprises an RV electrode proximate a portion of the patient's right ventricle (RV), and wherein monitoring electrical activity using the one or more electrodes during delivery of the LBB pacing at the one or more pacing configurations comprises monitoring electrical activity using the RV electrode proximate to the RV.

Example Ex4: the implantable medical device as in Example Ex1 or the method as in Example Ex2, wherein monitoring electrical activity using the one or more electrodes during delivery of the LBB pacing at the one or more pacing configurations comprises monitoring electrical activity using the LBB electrode proximate to the LBB.

Example Ex5: the implantable medical device or the method as in any one of Examples Ex1-Ex4, wherein the one or more pacing configurations comprises a plurality of pacing configurations,
wherein the computing apparatus is further configured to execute, or the method further comprises:
selecting a pacing configuration of the plurality of pacing configurations based on the surrogate LV activation time for each of the plurality of pacing configurations and a paced setting of each of the plurality of pacing configurations; and
delivering LBB pacing at the selected pacing configuration.

Example Ex6: the implantable medical device or the method as in Example Ex5, wherein selecting a pacing configuration of the plurality of pacing configurations based on the surrogate LV activation time for each of the plurality of pacing configurations and a paced setting of each of the plurality of pacing configurations further comprises:
selecting a further one or more pacing configurations of the plurality of pacing configurations, wherein the further one or more pacing configurations are each determined as capturing the LBB based on the determined surrogate LV activation time; and
selecting one of the further one or more pacing configurations of the plurality of pacing configurations, wherein the one pacing configuration comprises the lowest pacing output of the further one or more pacing configurations, and wherein the one pacing configuration is the selected pacing configuration.

Example Ex7: the implantable medical device or the method as in any one of Examples Ex1-Ex6, wherein the surrogate LV activation time extends between a LBB pacing pulse of the delivered LBB pacing and a fiducial point within the monitored electrical activity.

Example Ex8: the implantable medical device or the method as in Example Ex7, wherein the fiducial point is a maximum slope of the monitored activity within a lookback time window, wherein the lookback time window precedes a maximum peak of the monitored electrical activity within a post-pace time window following the LBB pacing pulse.

Example Ex9: the implantable medical device or the method as in Example Ex8, wherein the lookback time window is less than or equal to 75 milliseconds (ms) and the post-pace time window is less than or equal to 350 ms.

Example Ex10: the implantable medical device or the method as in Example Ex7, wherein the fiducial point is a maximum peak of the monitored activity within a post-pace time window following the LBB pacing pulse.

Example Ex11: the implantable medical device or the method as in Example Ex10, wherein the post-pace time window is less than or equal to 350 ms.

Example Ex12: the implantable medical device or the method as in any one of Examples Ex1-Ex11, wherein determining whether the LBB pacing has captured the LBB for each of the one or more pacing configurations comprises determining that the LBB pacing has captured the LBB if the surrogate LV activation time is within a selected percentage of one or more surrogate LV activation times determined from electrical activity monitored during delivery of LBB pacing using other pacing configurations of the one or more pacing configurations.

Example Ex13: the implantable medical device or the method as in Example Ex12, wherein the selected percentage is 20%.

Example Ex14: the implantable medical device or the method as in any one of Examples Ex12-Ex13, wherein the one or more surrogate LV activation times determined using the other pacing configurations of the one or more pacing configurations comprises a previously determined surrogate LV activation time that was determined from electrical activity monitored during delivery of LBB pacing using a most recently used pacing configuration.

Example Ex15: the implantable medical device or the method as in any one of Examples Ex1-Ex11, wherein determining whether the LBB pacing has captured the LBB for each of the one or more pacing configurations comprises determining that the LBB pacing has captured the LBB if the determined surrogate LV activation time is within a selected percentage of a threshold surrogate LV activation time.

Example Ex16: the implantable medical device or the method as in Example Ex15, wherein threshold surrogate LV activation time is determined from electrical activity of a template paced heartbeat.

Example Ex17: the implantable medical device or the method as in any one of Examples Ex15-Ex16, wherein the selected percentage is 20%.

Example Ex 18: the implantable medical device or the method as in any one of Examples Ex1-Ex11, wherein determining whether the LBB pacing has captured the LBB for each of the one or more pacing configurations comprises determining that the LBB pacing has captured the LBB if the surrogate LV activation time is within a selected percentage of one or more surrogate LV activation times determined from electrical activity monitored during delivery of LBB pacing using the same pacing configuration of the one or more pacing configurations.

Example Ex19: the implantable medical device or the method as in Example Ex18, wherein the selected percentage is 20%.

Example Ex20: the implantable medical device or the method as in any one of Examples Ex18-Ex19, wherein the one or more surrogate LV activation times determined using the same pacing configuration of the one or more pacing configurations comprises a previously determined surrogate LV activation time that was determined from electrical activity monitored during delivery of LBB pacing using a most recent heartbeat.

This disclosure has been provided with reference to illustrative embodiments and examples and is not meant to be construed in a limiting sense. As described previously, one skilled in the art will recognize that other various illustrative applications may use the techniques as described herein to take advantage of the beneficial characteristics of the devices and methods described herein. Various modifications of the illustrative embodiments and examples will be apparent upon reference to this description.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

All references and publications cited herein are expressly incorporated herein by reference in their entirety for all purposes, except to the extent any aspect directly contradicts this disclosure.

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

The recitation of numerical ranges by endpoints includes all numbers subsumed within that range (e.g., 1 to 5 includes 1, 1.5, 2, 2.75, 3, 3.80, 4, and 5) and any range within that range. Herein, the terms “up to” or “no greater than” a number (e.g., up to 50) includes the number (e.g., 50), and the term “no less than” a number (e.g., no less than 5) includes the number (e.g., 5).

The terms “coupled” or “connected” refer to elements being attached to each other either directly (in direct contact with each other) or indirectly (having one or more elements between and attaching the two elements). Either term may be modified by “operatively” and “operably,” which may be used interchangeably, to describe that the coupling or connection is configured to allow the components to interact to carry out at least some functionality (for example, a mobile user device may be operatively coupled to a cellular network transmit data to or receive data therefrom).

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

As used herein, “have,” “having,” “include,” “including,” “comprise,” “comprising” or the like are used in their open-ended sense, and generally mean “including, but not limited to.” It will be understood that “consisting essentially of,” “consisting of,” and the like are subsumed in “comprising,” and the like.

The term “and/or” means one or all of the listed elements or a combination of at least two of the listed elements.

The phrases “at least one of,” “comprises at least one of,” and “one or more of” followed by a list refers to any one of the items in the list and any combination of two or more items in the list.

1. An implantable medical device comprising:
one or more implantable electrodes comprising an LBB electrode positionable proximate a portion of a patient's left bundle branch (LBB); and
a computing apparatus comprising processing circuitry and operably coupled to the one or more implantable electrodes, wherein the computing apparatus is configured to:
deliver LBB pacing proximate to the LBB using the LBB electrode at one or more pacing configurations;
monitor electrical activity using the one or more implantable electrodes during delivery of the LBB pacing at the one or more pacing configurations;
determine a surrogate LV activation time based on the monitored electrical activity for each of the one or more pacing configurations, wherein the surrogate LV activation time is representative of an LV activation time; and
determine whether the LBB pacing has captured the LBB for each of the one or more pacing configurations based on the determined surrogate LV activation time for each of the one or more pacing configurations.

2. (canceled)

3. The implantable medical device of claim 1, wherein the one or more implantable electrodes further comprises an RV electrode proximate a portion of the patient's right ventricle (RV), and wherein monitoring electrical activity using the one or more electrodes during delivery of the LBB pacing at the one or more pacing configurations comprises monitoring electrical activity using the RV electrode proximate to the RV.

4. The implantable medical device of claim 1, wherein monitoring electrical activity using the one or more electrodes during delivery of the LBB pacing at the one or more pacing configurations comprises monitoring electrical activity using the LBB electrode proximate to the LBB.

5. The implantable medical device of claim 1, wherein the one or more pacing configurations comprises a plurality of pacing configurations,
wherein the computing apparatus is further configured to:
select pacing configuration of the plurality of pacing configurations based on the surrogate LV activation time for each of the plurality of pacing configurations and a paced setting of each of the plurality of pacing configurations; and
deliver LBB pacing at the selected pacing configuration.

6. The implantable medical device of claim 5, wherein selecting a pacing configuration of the plurality of pacing configurations based on the surrogate LV activation time for each of the plurality of pacing configurations and a paced setting of each of the plurality of pacing configurations further comprises:
selecting a further one or more pacing configurations of the plurality of pacing configurations, wherein the further one or more pacing configurations are each determined as capturing the LBB based on the determined surrogate LV activation time; and
selecting one of the further one or more pacing configurations of the plurality of pacing configurations, wherein the one pacing configuration comprises the lowest pacing output of the further one or more pacing configurations, and wherein the one pacing configuration is the selected pacing configuration.

7. The implantable medical device of claim 1, wherein the surrogate LV activation time extends between a LBB pacing pulse of the delivered LBB pacing and a fiducial point within the monitored electrical activity.

8. The implantable medical device of claim 7, wherein the fiducial point is a maximum slope of the monitored activity within a lookback time window, wherein the lookback time window precedes a maximum peak of the monitored electrical activity within a post-pace time window following the LBB pacing pulse.

9. The implantable medical device of claim 8, wherein the lookback time window is less than or equal to 75 milliseconds (ms) and the post-pace time window is less than or equal to 350 ms.

10. The implantable medical device of claim 7, wherein the fiducial point is a maximum peak of the monitored activity within a post-pace time window following the LBB pacing pulse.

11. The implantable medical device of claim 10, wherein the post-pace time window is less than or equal to 350 ms.

12. The implantable medical device of claim 1, wherein determining whether the LBB pacing has captured the LBB for each of the one or more pacing configurations comprises determining that the LBB pacing has captured the LBB if the surrogate LV activation time is within a selected percentage of one or more surrogate LV activation times determined from electrical activity monitored during delivery of LBB pacing using other pacing configurations of the one or more pacing configurations.

13. The implantable medical device of claim 12, wherein the selected percentage is 20%.

14. The implantable medical device of claim 12, wherein the one or more surrogate LV activation times determined using the other pacing configurations of the one or more pacing configurations comprises a previously determined surrogate LV activation time that was determined from electrical activity monitored during delivery of LBB pacing using a most recently used pacing configuration.

15. The implantable medical device of claim 1, wherein determining whether the LBB pacing has captured the LBB for each of the one or more pacing configurations comprises determining that the LBB pacing has captured the LBB if the determined surrogate LV activation time is within a selected percentage of a threshold surrogate LV activation time.

16. The implantable medical device of claim 15, wherein threshold surrogate LV activation time is determined from electrical activity of a template paced heartbeat.

17. The implantable medical device of claim 15, wherein the selected percentage is 20%.

18. The implantable medical device of claim 1, wherein determining whether the LBB pacing has captured the LBB for each of the one or more pacing configurations comprises determining that the LBB pacing has captured the LBB if the surrogate LV activation time is within a selected percentage of one or more surrogate LV activation times determined from electrical activity monitored during delivery of LBB pacing using the same pacing configuration of the one or more pacing configurations.

19. The implantable medical device of claim 18, wherein the selected percentage is 20%.

20. The implantable medical device of claim 18, wherein the one or more surrogate LV activation times determined using the same pacing configuration of the one or more pacing configurations comprises a previously determined surrogate LV activation time that was determined from electrical activity monitored during delivery of LBB pacing using a most recent heartbeat.

21. A method comprising:
delivering LBB pacing proximate to a patient's left bundle branch (LBB) using an LBB electrode positioned proximate a portion of the LBB at one or more pacing configurations;
monitoring electrical activity using one or more electrodes during delivery of the LBB pacing at the one or more pacing configurations;
determining a surrogate LV activation time based on the monitored electrical activity for each of the one or more pacing configurations, wherein the surrogate LV activation time is representative of an LV activation time; and
determining whether the LBB pacing has captured the LBB for each of the one or more pacing configurations based on the determined surrogate LV activation time for each of the one or more pacing configurations.

22. The method of claim 21, wherein monitoring electrical activity using the one or more electrodes during delivery of the LBB pacing at the one or more pacing configurations comprises monitoring electrical activity using an RV electrode proximate a portion of the patient's right ventricle (RV).

23. The method of claim 21, wherein monitoring electrical activity using the one or more electrodes during delivery of the LBB pacing at the one or more pacing configurations comprises monitoring electrical activity using the LBB electrode proximate to the LBB.

24. The method of claim 21, wherein the one or more pacing configurations comprises a plurality of pacing configurations,
wherein the method further comprises:
selecting a pacing configuration of the plurality of pacing configurations based on the surrogate LV activation time for each of the plurality of pacing configurations and a paced setting of each of the plurality of pacing configurations; and
delivering LBB pacing at the selected pacing configuration.

25. The method of claim 21, wherein the surrogate LV activation time extends between a LBB pacing pulse of the delivered LBB pacing and a fiducial point within the monitored electrical activity.

26. The method of claim 21, wherein determining whether the LBB pacing has captured the LBB for each of the one or more pacing configurations comprises determining that the LBB pacing has captured the LBB if the surrogate LV activation time is within a selected percentage of one or more surrogate LV activation times determined from electrical activity monitored during delivery of LBB pacing using other pacing configurations of the one or more pacing configurations.

27. The method of claim 21, wherein determining whether the LBB pacing has captured the LBB for each of the one or more pacing configurations comprises determining that the LBB pacing has captured the LBB if the determined surrogate LV activation time is within a selected percentage of a threshold surrogate LV activation time.

28. The method of claim 21, wherein determining whether the LBB pacing has captured the LBB for each of the one or more pacing configurations comprises determining that the LBB pacing has captured the LBB if the surrogate LV activation time is within a selected percentage of one or more surrogate LV activation times determined from electrical activity monitored during delivery of LBB pacing using the same pacing configuration of the one or more pacing configurations.
